(19) United States
(12) Patent Application Publication
(10) Pub. No.: US 2026/0035718 A1
(43) Pub. Date: Feb. 5, 2026

(54) BIOLFILM BIOREACTOR INCLUDING THERMAL MANAGEMENT AND SUBSTANCE DELIVERY ELEMENTS, RELATED APPARATUS, AND RELATED METHODS

(71) Applicant: REGENTS OF THE UNIVERSITY OF MICHIGAN, Ann Arbor, MI (US)

(72) Inventors: Timothy Fairley-Wax, Ann Arbor, MI (US); Steven Skerlos, Ann Arbor, MI (US); Lutgarde Raskin, Ann Arbor, MI (US); Katherine Giammalvo, Columbia, MD (US); Carla Voigt, Kihei, HI (US); Rolland Barnum, Clarendon Hills, IL (US); Liam Maloney, Grand Rapids, MI (US)

(21) Appl. No.: 18/873,802

(22) PCT Filed: Jun. 20, 2023
(86) PCT No.: PCT/US2023/025700

§ 371 Date: Dec. 11, 2024

Related U.S. Application Data
(60) Provisional application No. 63/353,701, filed on Jun. 20, 2022.

(51) Int. Cl. C12P 5/02; C12M 1/00; C12M 1/107; C12M 1/12; C12M 1/34

(52) U.S. Cl. CPC C12P 5/023; C12M 21/04; C12M 23/40; C12M 25/02; C12M 29/04; C12M 41/16

(57) ABSTRACT

The disclosure relates to bioreactors, for example for biological treatment and, more specifically to bioreactor insert apparatus including temperature control and substance delivery elements. The temperature control elements include conduits to circulate a thermal or working fluid and provide a means to locally maintain a biofilm support and biofilm thereon at a temperature promoting growth of the biofilm microorganisms. The conduits for temperature control can further serve as substance delivery elements, providing a means for selective transport and biofilm-localized delivery of growth-promoting substances in the circulating thermal fluid. The temperature control and substance delivery elements can increase the overall reaction rate of the bioreactor.

### CROSS REFERENCE TO RELATED APPLICATION

This application claims the benefit under 35 U.S.C. § 119(e) of U.S. Provisional Patent Application No. 63/353,701, filed Jun. 20, 2022, the entire disclosure of which is incorporated herein by reference.

### STATEMENT OF GOVERNMENT INTEREST

None.

### FIELD OF THE DISCLOSURE

The present disclosure relates to bioreactors, for example for biological treatment and, more specifically to bioreactor insert apparatus including temperature control and substance delivery elements, bioreactors incorporating the bioreactor insert apparatus with biofilms thereon, and related methods.

### BACKGROUND

Membrane bioreactors (MBRs) are used in a wide array of filter applications. MBRs combine microbial processes in a bioreactor containing diverse microbial communities or specific microorganisms with a membrane barrier that filters out the microorganisms from the water in the bioreactor.

MBRs, which employ microfiltration or ultrafiltration membranes, have attracted considerable attention in the wastewater treatment industry due to their ability to produce a high-quality effluent at reasonable hydraulic retention times (HRTs), and anaerobic MBRs (AnMBRs) also produce energy in the form of biogas. Anaerobic digestion is a process which utilizes bacteria to break down organic matter without oxygen. This process is commonly used in wastewater treatment and is useful for a variety of organic matter including manure, municipal sewage, food waste, and other components that are common in wastewater streams. Anaerobic digestion creates two byproducts: biogas and digestate. Biogas is composed primarily of methane, and may also contain other gases like carbon dioxide, hydrogen sulfide, water vapor, etc. This biogas can be captured and used as an energy source or processed into various biochemical products. The term digestate refers to the solid and liquid byproducts of the anaerobic digestion process. Digestate is nutrient rich and can be processed further to create a variety of useful products.

There are many methods of anaerobic digestion. Methods are selected based on the scale of the system and the properties of the influent wastewater. These methods can be grouped into two categories. The first type of anaerobic wastewater system is a suspended growth system. This type of system relies on microorganisms that are mixed in with the wastewater, and often requires a very large hydraulic retention time. Hydraulic retention time is a representation of the average time that fluid stays inside the reactor. Systems with a larger HRT are often more costly to operate, since they require a much larger system volume to treat the same volume of water. The second type of anaerobic digester is an attached growth system. These systems allow the microorganisms to grow in a film on some type of support media. There are many kinds of media that can be used in these systems.

However, even AnMBRs are not currently advantageous for treatment of low to medium strength wastewater relative to current high rate activated sludge (aerobic) systems which employ anaerobic treatment of sludge, from net energy usage and net global warming impact perspectives. Two major drawbacks such as high energy requirement for membrane fouling mitigation and expensive membrane modules, which limit the scale at which MBRs can be applied, are solved by shifting to dynamic membrane bioreactors (DMBRs). This is because DMBRs employ coarser meshes (larger pore size than the membranes used in MBRs) made from materials such as nylon, silk, or stainless steel which are less energy intensive to clean and much cheaper to produce. Additionally, DMBRs can operate at higher fluxes than MBRs, which means that low HRTs (high loading rates), which are essential to achieve high methane yields during anaerobic treatment of low to medium strength wastewater, can be achieved with even smaller physical footprint. Current DMBRs employ a similar membrane configuration to conventional MBRs (i.e., a tubular or flatsheet membrane in a submerged or external side-stream configuration), and thus also have significant global warming impact, largely due to dissolved methane in reactor effluent. Dynamic membranes are typically applied in continually stirred tank reactors (CSTRs) or upflow anaerobic sludge blanket reactors (UASBs), and the system relies on biological treatment performance in the reactor and any additional treatment from the biofilm that forms on the mesh (referred to as a dynamic membrane). Previous studies with AnMBRs have shown that biofilms become increasingly important for organics removal relative to the biomass in suspension in the bioreactor as wastewater temperature decreases. Similarly, biofilms in aerobic MBRs are also important for treatment when high quality effluents are needed. Adequate treatment results have already been achieved by both aerobic and anaerobic DMBRs in lab settings, and now at pilot-scale for treatment of both low and high strength wastewaters. Currently, there are not any commercial dynamic membrane modules, and all studies have used self-made modules.

CN 104045170A discloses a sewage treatment device based on 3D printing of biological biofilm carrier media. The apparatus includes plastic biofilm carrier media that is specifically designed to have a large surface area and a roughness so that biofilm adhesion is faster. The 3D-printed media is suspended in the reactor which treats wastewater. The design uses 3D printing technology to enhance surface area and allow biofilm formation for treatment of wastewater.

US 2021/0309955 is directed to bioreactors, a bioreactor insert and biofilm support, related apparatus, and related methods. The bioreactor insert apparatus provides a means for circulation of reaction medium within the bioreactor, a biofilm support, and biological treatment of an inlet feed to the reactor/insert apparatus. The bioreactor insert apparatus has a high relative surface area for biofilm attachment and is capable of generating complex flow patterns and increasing treatment efficiency/biological conversion activity in a biologically-active reactor.

One of the major challenges facing membrane bioreactor technology is the low temperature of most municipal wastewater. Microorganisms exhibit their highest growth rates and overall metabolic performance at their temperature optima. The type of bacteria which are most useful in anaerobic digestion are called mesophiles. Mesophiles are microorganisms that have temperature optima in the range of 25 to 40° C. By contrast, ambient water temperatures for influent wastewater can often be as low as 10° C. Mesophiles have substantially higher growth rates than psychrophiles, microorganisms with temperature optima in the range of 5 to 15° C. Higher growth rates result in faster biochemical conversion rates, which is useful in wastewater treatment and industrial fermentation applications. Temperature control also enables selection of specific microorganisms, which can be beneficial for promoting specific metabolic pathways.

Temperature control in wastewater treatment is conventionally accomplished by pumping the entirety of the bulk medium through a heat exchanger (or similar) and back to the reaction vessel (i.e., a bioreactor). In some industrial fermentation applications, jacketed bioreactors (i.e., fermenters) use a recirculated coolant (e.g., glycol) or heating fluid (e.g., oil) accompanied by a chiller/heat source to accomplish temperature control. These methods require a large energy input to provide heat to microorganisms.

### SUMMARY

In one aspect, the disclosure relates to a bioreactor insert apparatus for reaction medium circulation, biofilm support, and biological treatment, the apparatus comprising: an inlet volume in fluid communication with a first fluid inlet; at least one exit volume (e.g., a plurality of exit volumes), each exit volume being in (direct or indirect) fluid communication with the inlet volume and comprising at least one exit biofilm support (e.g., mesh) disposed at a boundary between the exit volume and an external volume outside the bioreactor insert apparatus, wherein: the exit biofilm support is adapted to promote growth, attachment, and metabolism of microorganisms in the form of a biofilm thereon, and the exit biofilm support is adapted to permit fluid and solid transport across the exit biofilm support and between the exit volume and the external volume; and at least one exit conduit mounted adjacent to the exit biofilm support. The conduit generally can include, for example, an exit conduit when mounted adjacent to an exit volume, a recirculation conduit when mounted adjacent to a recirculation volume as described below, etc. The conduit can include a pipe, tube or other structure for flow of a thermal or other working fluid/liquid for temperature control and/or substance delivery. The adjacent spatial relationship between the conduit and the biofilm support (or biofilm) can include arrangements in which the conduit is on or in direct physical contact with the biofilm support and/or biofilm. Alternatively, conduit can be substantially close to the biofilm support (e.g., within 1, 2, 4, 6, 8, or 10 mm), for example spaced apart from the biofilm support such that growth of the biofilm thereon would provide contact or closer proximity of the biofilm and the conduit. Proximity between the conduit and the biofilm support or biofilm (if not direct contact) is preferably close enough to permit sufficient conductive heat transfer to maintain the biofilm support and/or biofilm at a desired temperature as described below for different microorganisms or other operating targets.

Each exit conduit comprises a thermal fluid inlet, a thermal fluid outlet, and an interior volume (e.g., volume between inlet/outlet and defined by conduit outer wall(s)) adapted to receive and permit flow of a thermal fluid (or working fluid, for example a liquid such as or including water) from the thermal fluid inlet to the thermal fluid outlet via the interior volume. The exit conduit interior volume is substantially liquidly isolated from the first fluid inlet, the inlet volume, the exit volume, and the external volume (e.g., and first fluid outlet, the outlet volume, and the recirculation volume when present). Substantial liquid isolation includes limiting or preventing liquid flow, such as less than 0.01, 0.1, 1, 2, or 5% of liquid volumetric or mass flow rate through exit conduit can pass through the exit conduit wall (e.g., a semi-permeable portion thereof) into the external environment. When the exit conduit includes a semi-permeable portion, gases, dissolved or ionic species, and/or small particulate solids (e.g., micro- or nano-scale) can pass from the exit conduit interior volume into the external environment, such as from the thermal fluid to the bulk reaction medium. When the exit conduit does not include a semi-permeable portion, the exit conduit interior volume can be completely isolated from the first fluid inlet, the inlet volume, the exit volume, and the external volume (e.g., and first fluid outlet, the outlet volume, and the recirculation volume when present), such that there is essentially no liquid, gas, or solid material passing from the thermal fluid to the bulk reaction medium. The exit conduit is adapted to provide heat transfer between a thermal fluid in the exit conduit and the biofilm support (e.g., and any biofilm thereon), thereby controlling a temperature of the biofilm support (e.g., and any biofilm thereon). In particular, controlling or selecting the temperature of the thermal fluid delivered to the exit conduit in turn permits control of the resulting temperature of the biofilm support or biofilm. Such heat transfer primarily includes conductive transport and optionally includes some convective transport, such as in reaction medium liquid passing through the biofilm and/or flow of reaction medium liquid in the vicinity of the biofilm/biofilm support; these transport effects can alternatively be collectively referenced as radiative heat transport. More specifically, heat transfer and temperature control can include a combination of heat conduction within the solid mesh or other biofilm support material as well as a conductive (and possibly convective) heat loss from the mesh or biofilm support into the bulk liquid reaction medium or external environment. The features described herein related to exit conduits can apply analogously to recirculation conduits (or other temperature-control/substance-delivery conduits) when present.

Various embodiments of the disclosed bioreactor insert apparatus are possible.

In an embodiment, the exit conduit comprises a metal material (e.g., formed from a metal such as steel, stainless steel, copper or other metal tubing). The conduit more generally can be formed from any suitable thermally conductive material to promote heat transfer between the thermal fluid and the biofilm support through the conduit wall. Conduit materials such as stainless steel can also be selected based on their ability to resist corrosion or other degradation due to exposure to the bulk bioreactor liquid medium. The conduit can be formed from such thermally conductive materials in regions where the conduit is in contact with/adjacent to the biofilm support, for example where the conduit also has regions where it is formed from thermally resistive materials to limit heat losses while the thermal fluid is transferred to/from the thermally conductive regions (e.g., to/from an external heater/cooler/pump for recirculation). For example, typical thermal conductivity values (e.g., at a reference temperature of 25° C.) for metals or other thermally conductive materials can range from 5 to 500 W/(m K), for example at least 5, 15, 30, 50, 100, 200, or 300 W/(m K) and/or up to 50, 100, 200, 300, 400, or 500 W/(m K).

In an embodiment, the exit conduit comprises a thermally insulating material for one or more regions of the exit conduit that are not adjacent to (e.g., not in contact with) the biofilm support. As noted above, the conduit can include thermally resistive tubing/piping etc. attached to the thermally conductive regions in contact with the biofilm support. In addition to using a non-metal plastic or other thermally resistive tubing for recirculation in non-heat conducting areas, the conduit can also incorporate a thermal insulation material as a partial sleeve or coating, etc. around a portion of the metal or other thermally conductive conduit material that is not in contact with or otherwise adjacent to the biofilm support. For example, 75% (or other selected fraction) of the circumference of the thermally conductive conduit that is generally in contact with the bulk liquid medium can be covered, coated, etc. with an insulating material to limit heat losses into the bulk liquid medium, while the remaining 25% (or other selected fraction) of the circumference of the thermally conductive conduit that is in contact with or directly adjacent (e.g., facing) the biofilm support or biofilm is bare to promote heat conduction at a metal-metal or other highly heat conductive interface. For example, typical thermal conductivity values (e.g., at a reference temperature of 25° C.) for plastics, rubbers, or other thermally resistive materials (e.g., for thermally resistive tubing or piping, thermally insulating sleeve or coating) can range from 0.001 to 5 W/(m K), for example at least 0.001, 0.01, 0.1, 0.2 or 0.5 W/(m K) and/or up to 0.1, 0.4, 0.7, 1, 2, 3, 4, or 5 W/(m K).

In an embodiment, the exit conduit comprises a semi-permeable material adapted to permit selective transport of a (non-liquid) substance in the thermal fluid through the semi-permeable material, for example into or in the immediate vicinity of the biofilm support or biofilm such that the transported substance can then promote, stimulate, regulate, or otherwise control growth of microorganisms in the biofilm. Such transport and delivery of the substance from the thermal fluid and through the semi-permeable material can be into one or more regions of the exit volume, the recirculation volume, the external volume, etc. that are sufficiently close to the target biofilm support or biofilm. The non-liquid substances for selective transport can include one or more of gases, dissolved or ionic species, and/or small particulate solids (e.g., micro- or nano-scale solids). Specific examples of substances for delivery include oxygen gas (e.g., with nitrogen gas as in air), trace metals, nitrogen or nitrogen-containing compounds (e.g., as a nutrient), phosphorous or phosphorous-containing compounds (e.g., as a nutrient), nanoparticles, bleach, sodium hydroxide, nutrients more generally, etc. Semi-permeable materials (e.g., semi-permeable membranes) are generally known in the art and are commercially available for a variety of selective transport target substances. For example, a commercially available OXYMEM membrane (available from DuPont) can be used to selectively transport and supply oxygen gas from air to a biofilm.

The semi-permeable material can be incorporated into the conduit in a variety of configurations. In some embodiments, the semi-permeable material can be formed from a thermally conductive material through which heat is conducted for temperature control. In some embodiments, the semi-permeable material can be in a separate region from that where heat transfer is occurring (e.g., a separate semi-permeable tubing connected to the copper or other heat conductive tubing). In some embodiments, a wall portion of a copper, metal, or other heat conductive conduit can be replaced with a semi-permeable material as a liquid-impermeable barrier that still permits selective substance transport and delivery. The semi-permeable material is generally positioned so that it delivers nutrients or other selectively transported substance spatially close to the biofilm support/biofilm.

In an embodiment, the exit conduit is in fluid communication with one or more of a heater, cooler, and pump for circulation of a temperature-controlled thermal fluid through the exit conduit. For example, the exit conduit can be in fluid communication with the heater and the cooler; and the exit conduit can be adapted to cycle between heating and cooling states in which relatively warmer and cooler thermal fluids are transported through exit conduit to expand and contract the biofilm support and any biofilm thereon (e.g., as part of a cleaning or other maintenance process for the bioreactor).

In an embodiment, the exit conduit is in fluid communication with a reservoir containing the thermal fluid therein. For example, the reservoir can include heating and/or cooling elements such that the reservoir also serves as the heater and/or cooler components described above. The reservoir can be fluidly connected to the pump for circulation. In various embodiments, the thermal fluid (e.g., in the reservoir or otherwise) can include one or more liquids selected for their heat transport properties, for example including water, glycols (e.g., ethylene glycol), oils, etc. In addition to the liquid providing the heat for temperature control, the thermal fluid additionally can contain any of the substances described above for selective transport and delivery to the biofilm.

In an embodiment, the exit conduit is adapted to maintain the biofilm support (e.g., and any biofilm thereon) at a temperature in a range of 25° C. to 40° C. More generally, suitable target temperatures can include those appropriate for mesophilic microorganisms in the biofilm, for example at least 25, 30, 32, or 35° C., up to 30, 35, 37, or 40° C., and/or not higher than 36° C., 37° C., 38° C., 39° C., or 40° C.

In an embodiment, the exit conduit is adapted to maintain the biofilm support (e.g., and any biofilm thereon) at a temperature in a range of 40° C. to 60° C. More generally, suitable target temperatures can include those appropriate for thermophilic microorganisms in the biofilm, for example at least 40, 45, 50, or 55° C., up to 45, 50, 55, or 60° C., and/or not higher than 56° C., 57° C., 58° C., 59° C., or 60° C.

In an embodiment, the exit conduit is adapted to maintain the biofilm support (e.g., and any biofilm thereon) at a temperature in a range of 60° C. to 100° C. More generally, suitable target temperatures can include those appropriate for hyperthermophilic microorganisms in the biofilm, for example at least 60, 65, 70, or 80° C., up to 70, 80, 90, or 100° C., and/or not higher than 80° C., 90° C., 95° C., 98° C., or 100° C.

In an embodiment, the exit conduit is adapted to maintain the biofilm support (e.g., and any biofilm thereon) at a temperature of at least 5° C. higher than a bulk liquid temperature in the exit volume and in the external volume. More generally, suitable target temperatures can be at least 5, 7, 10, 20, 30, 40, 50, or 60° C. higher and/or up to 10, 12, 15, 20, 30, 40, 50, 60, 80, or 95° C. higher than the bulk liquid temperature to promote one or more of mesophilic, thermophilic, or hyperthermophilic microorganism growth.

In an embodiment, the exit conduit is adapted to maintain the biofilm support (e.g., and any biofilm thereon) at a temperature in a range of 1° C. to 20° C. More generally, suitable target temperatures can include those appropriate for psychrophilic microorganisms in the biofilm, for example at least 1, 2, 5, 7, or 10° C., and/or up to 6, 8, 10, 12, 15, or 20° C.

In an embodiment, the exit conduit is adapted to maintain the biofilm support (e.g., and any biofilm thereon) at a temperature of at least 5° C. lower than a bulk liquid temperature in the exit volume and in the external volume. More generally, suitable target temperatures can be at least 5, 7, 10, or 12° C. lower and/or up to 8, 10, 12, 15, or 20° C. lower than the bulk liquid temperature to promote psychrophilic microorganism growth.

In an embodiment, the exit conduit is adapted to maintain the biofilm support (e.g., and any biofilm thereon) at a temperature in a range of 15° C. to 30° C. More generally, suitable target temperatures can be at least 15, 20, or 25° C., and/or up to 20, 25, or 30° C.

In an embodiment, the exit conduit is adapted to maintain the biofilm support (e.g., a portion of or substantially the entire biofilm support and any biofilm thereon) at a temperature within ±6° C. of a selected setpoint temperature. Typical temperature variation windows accounting for lateral and longitudinal variations in heat transfer within the biofilm support and/or biofilm can be ±0.5, 1, 1.5, 2, 2.5, 3, 3.5, 4, 5, or 6° C. of a selected setpoint temperature. Suitable setpoint temperatures can be selected based on the psychrophilic, mesophilic thermophilic, or hyperthermophilic character of the desired microorganisms in the biofilm, for example including setpoints or 4, 6, 8, 10, 12, or 14° C., for psychrophilic microorganisms; 29, 31, 33, 35, 37, or 39° C., for mesophilic microorganisms; 44, 48, 52, 56, or 58° C., for thermophilic microorganisms; or 64, 68, 74, 80, 86, 92, or 98° C., for hyperthermophilic microorganisms.

In an embodiment, the spatial temperature distribution of the biofilm support (e.g., and any biofilm thereon) can be suitably narrow such that the substantial portion of the biofilm experiences a consistent desired temperature that maximizes its growth while avoiding extreme temperatures that can kill the microorganisms and/or result in lower growth rates. The foregoing temperature ranges, windows, and setpoints can apply to a portion of or substantially the entire biofilm support and any biofilm thereon, such as at least 30, 50, 70, 80, 90, 95, 98, or 99% and/or up to 60, 75, 85, 95, 98, 99, or 100% of the biofilm support/biofilm volume or surface area has the desired temperature range, window, and/or setpoint. The spatial temperature distribution can be determined using a series of spatially distributed thermocouples or other temperature measurement apparatus, computational heat transfer modeling, etc. The threshold of biofilm support/biofilm area or volume accounts for possible small peripheral areas that might not achieve the desired temperature, but the desired temperature facilitating robust microorganism growth can be obtained over a substantial fraction of the biofilm support/biofilm.

In an embodiment, the exit conduit is adapted to maintain a temperature gradient in the biofilm support (e.g., and any biofilm thereon) with at least two different setpoint temperatures that are at least 2° C. different from each other. For example, the different setpoint temperatures that are at least 2, 3, 5, 7, 10, 15, 20, or 25° C., and/or up to 4, 6, 8, 10, 12, 15, 20, 30, or 40° C. different from each other. The different setpoint temperatures can correspond to two or more exit conduits or segments thereof in contact with biofilm support or biofilm, which conduits or segments transport thermal fluids having similar temperature differences as described for the setpoints.

In an embodiment, each biofilm support is adjacent to (e.g., in direct contact with) a plurality of exit conduit segments at spaced-apart locations of the biofilm support. This structure can minimize/reduce lateral and/or longitudinal temperature gradients in the biofilm support and corresponding biofilm by including multiple conduit segments at multiple different lateral and/or longitudinal locations of the biofilm support, for example where the different conduit segments contain thermal fluids at substantially the same temperature. In other cases, this structure can induce or control desired lateral and/or longitudinal temperature gradients in the biofilm support and corresponding biofilm by including multiple conduit segments at multiple different lateral and/or longitudinal locations of the biofilm support, for example where the different conduit segments contain thermal fluids at different temperatures. Separate conduit segments can include (i) separate conduit tubes in a parallel arrangement from the same or different heating/cooling/pump reservoir(s), and/or (ii) different regions of a single conduit tube that winds/wraps around the biofilm support at different locations.

In an embodiment, the thermal conductivity along the length of the conduit tube can be varied so that there is more insulation/resistance to heat conduction at the inlet vs. the outlet such that comparatively hotter thermal fluid at the inlet still results in the same or similar biofilm support/biofilm temperature as compared to colder thermal fluid at the outlet (e.g., to reduce longitudinal temperature gradients in the biofilm support/biofilm). For example, a thin intermediate insulating material that reduces in thickness from the inlet to the outlet can be incorporated between the conduit and the biofilm support/biofilm. In other case, the thermal conductivity along the length of the conduit tube can similarly be selected or varied to induce a desired temperature gradient in the biofilm support/biofilm.

In an embodiment, the exit conduit is positioned at least partially within the exit volume. This can represent an embodiment in which the conduit is on the inside of the biofilm support or mesh, for example providing structural support for the biofilm support and constituting a part of the exit volume or recirculation volume structure.

In an embodiment, the exit conduit is positioned at least partially within the external volume. This can represent an embodiment in which the conduit is on the outside of the biofilm support or mesh, for example providing structural support for the biofilm support and constituting a part of the exit volume or recirculation volume structure.

In an embodiment, the exit conduit is positioned at least partially within the exit volume and the external volume. This can represent an embodiment in which the conduit is integrally formed with the biofilm support or mesh, for example where smaller lattice or mesh elements span and are affixed to/integral with adjacent conduit tubes, providing structural support for the biofilm support and constituting a part of the exit volume or recirculation volume structure.

In an embodiment, the bioreactor insert apparatus further comprises: an outlet volume in fluid communication with a first fluid outlet; at least one recirculation volume (e.g., a plurality of recirculation volumes), each recirculation volume being in fluid communication with the outlet volume and comprising at least one recirculation biofilm support disposed at a boundary between the recirculation volume and the external volume outside the bioreactor insert apparatus, wherein: the recirculation biofilm support is adapted to promote growth, attachment, and metabolism of microorganisms in the form of a biofilm thereon, and the recirculation biofilm support is adapted to permit fluid and solid transport across the recirculation biofilm support and between the recirculation volume and the external volume; and optionally at least one recirculation conduit mounted adjacent to the recirculation biofilm support. Each recirculation conduit comprises a thermal fluid inlet, a thermal fluid outlet, and an interior volume adapted to receive and permit flow of a thermal fluid from the thermal fluid inlet to the thermal fluid outlet via the interior volume; the recirculation conduit interior volume is substantially liquidly isolated from the first fluid inlet, the first fluid outlet, the inlet volume, the outlet volume, the exit volume, the recirculation volume, and the external volume; and the recirculation conduit is adapted to provide heat transfer between a thermal fluid in the recirculation conduit and the recirculation biofilm support, thereby controlling a temperature of the recirculation biofilm support.

In an embodiment, the bioreactor insert apparatus comprises a plurality of the exit volumes, wherein the exit biofilm support is the only fluid communication pathway between the exit volume and the external volume, such that the exit volume is configured to force circulating liquid from the inlet volume, through the exit biofilm support, and into the external volume; and a plurality of the recirculation volumes, wherein the recirculation biofilm support is the only fluid communication pathway between the recirculation volume and the external volume, such that the recirculation volume is configured to receive forced circulating liquid from the external volume, through the recirculation biofilm support, and into the outlet volume.

In an embodiment, the bioreactor insert apparatus is a single structure comprising the inlet volume, the outlet volume, the plurality of exit volumes, and the plurality of recirculation volumes; the inlet volume is defined by a cylindrical tube in fluid communication with the first fluid inlet; the outlet volume is defined by an annular tube around the cylindrical tube in fluid communication with the first fluid outlet; the exit volumes are defined by exit tubes in fluid communication with the cylindrical tube inlet volume; and the recirculation volumes are defined by recirculation tubes in fluid communication with the annular tube outlet volume.

In an embodiment, the biofilm support is in the form of a mesh.

In an embodiment, the bioreactor insert apparatus further comprises a biofilm adhered to the biofilm support, the biofilm comprising a community of microorganisms collectively having biological activity.

In an embodiment, the bioreactor insert apparatus further comprises a biofilm seed adhered to the biofilm support, the biofilm seed comprising a water-soluble adhesive matrix and a community of microorganisms as a biofilm precursor distributed throughout the matrix.

In another aspect, the disclosure relates to a bioreactor comprising: a reaction vessel defining an interior reaction volume; and a bioreactor insert apparatus according to any of their variously disclosed embodiments mounted within the reaction vessel; wherein the external volume corresponds to a portion of the interior reaction volume outside the bioreactor insert apparatus

In another aspect, the disclosure relates to a method for forming a bioreactor product (e.g., methane), the method comprising: providing a bioreactor and bioreactor insert apparatus according to any of their variously disclosed embodiments, wherein: the bioreactor insert apparatus further comprises biofilms adhered to the biofilm supports, the biofilms having biological (e.g., methanogenic) activity, an aqueous reaction medium at least partially fills the interior reaction volume and the bioreactor insert apparatus, and suspended microorganisms are present in the aqueous reaction medium; feeding a thermal fluid (or working fluid) having a selected (e.g., controlled) temperature through the conduits mounted adjacent to the biofilm supports (e.g., exit conduits adjacent to the exit biofilm supports, recirculation conduits adjacent to the recirculation biofilm supports, etc.), thereby maintaining (or controlling) the temperature of the biofilm supports and biofilms thereon at a pre-selected temperature or within a temperature range (e.g., a substantially uniform temperature or an induced temperature gradient); feeding an influent stream (e.g., wastewater influent) comprising one or more reactants for conversion to the bioreactor insert apparatus via the first fluid inlet; circulating the influent stream through the inlet volume, into the exit volumes, forced through the exit biofilm supports and biofilms thereon, into the external volume, forced through the recirculation biofilm supports and biofilms thereon, into the recirculation volumes, into the outlet volume, and through the first fluid outlet; and converting the one or more influent reactants to a (e.g., methane) product, in particular by biofilm activity and/or suspended microorganism activity.

Various embodiments of the disclosed method are possible, for example including the bioreactor insert apparatus and/or bioreactor in any of their variously disclosed embodiments.

In an embodiment, the thermal fluid further comprises a substance (e.g., gas, dissolved species, small particulate solid in addition to the transporting liquid medium) for delivery to the biofilm; at least one conduit comprises a semi-permeable material adapted to permit selective transport of the substance from the thermal fluid through the semi-permeable material; and delivering (or transporting) the substance to the biofilm through the semi-permeable material.

In an embodiment, at least 50%, 65%, or 80% of total microorganisms in the bioreactor are incorporated into the biofilms.

In an embodiment, the biofilms have methanogenic activity.

In an embodiment, the biofilms comprising a community of anaerobic microorganisms.

In an embodiment, the biofilms comprising a community of aerobic microorganisms.

In an embodiment, the biofilms comprising a community of anoxic microorganisms.

In an embodiment, the method comprises operating the bioreactor at a temperature in a range of 5° C. to 30° C., 15° C. to 30° C., or 20° C. to 25° C. These temperatures can represent the ambient or bulk fluid temperature of the bioreactor, such as the temperature of the influent stream and/or the temperature of the liquid reaction medium in the interior reaction volume or the reaction vessel, for example including a range of time-dependent operating temperatures resulting from (naturally) varying influent temperatures (e.g., in which cases the thermal management conduits can still maintain desired biofilm/biofilm support operating temperatures).

While the disclosed apparatus, methods, and compositions are susceptible of embodiments in various forms, specific embodiments of the disclosure are illustrated (and will hereafter be described) with the understanding that the disclosure is intended to be illustrative, and is not intended to limit the claims to the specific embodiments described and illustrated herein.

### BRIEF DESCRIPTION OF THE DRAWINGS

FIG. 1 illustrates a bioreactor insert apparatus and corresponding bioreactor according to the disclosure, which apparatus and bioreactor can treat aqueous wastewater comprising organic components by sending the wastewater through multiple exit and recirculation volumes in series separated by meshes or supports that serve for biofilm attachment.

FIG. 2 is a cut-away perspective view of a bioreactor insert apparatus according to the disclosure.

FIG. 3 is a cut-away side view of a bioreactor insert apparatus according to the disclosure.

FIG. 4 is a perspective view of a bioreactor insert apparatus according to the disclosure and illustrating an increased specific surface area for biofilm supports/biofilms.

FIG. 5 illustrates a bioreactor insert apparatus and corresponding bioreactor according to the disclosure in an embodiment in which the insert apparatus has separate influent and effluent structures. Panel (A) illustrates an embodiment with the influent and effluent structures in the same bioreactor vessel. Panel (B) illustrates an embodiment with the influent and effluent structures in separate bioreactor vessels that collectively define the bioreactor.

FIG. 6 is a partial view of a bioreactor insert apparatus according to the disclosure illustrating non-limiting configurations of the exit conduit and recirculation conduit.

FIG. 7A is a partial view of a bioreactor insert apparatus according to the disclosure illustrating a non-limiting configuration of a conduit, thermal fluid inlet, and thermal fluid outlet.

FIG. 7B is a cross-sectional view of the exit/recirculation volume illustrated in FIG. 7A, illustrating a plurality of conduit/conduit internal volumes integrally provided within the biofilm support or mesh.

FIG. 7C is a cross-sectional view of an exit/recirculation volume of the disclosure, illustrating a plurality of conduit/conduit internal volumes positioned at least partially within the exit volume and/or recirculation volume. The conduit is on the inside of the biofilm support or mesh, for example providing structural support for the biofilm support and constituting a part of the exit volume or recirculation volume structure.

FIG. 7D illustrates a non-limiting configuration for a plurality of conduits to provide structural support for the biofilm support and constituting a part of the exit volume or recirculation volume.

FIG. 8 is a partial view of a bioreactor insert apparatus according to the disclosure illustrating a non-limiting configuration of a conduit, thermal fluid inlet, and thermal fluid outlet.

FIG. 9 illustrates a bioreactor insert apparatus according to the disclosure, which apparatus can treat aqueous wastewater comprising organic components by sending the wastewater from outside the apparatus, through multiple biofilms/biofilm supports, to the inside of a central channel.

FIG. 10 is a top-down view of one layer of the bioreactor insert apparatus of FIG. 9. The branches comprise three conduits surrounded by biofilm support. The wastewater enters the apparatus through the biofilm support and is fed into the central column.

FIG. 11 is a diagram showing the setup for the mathematical model used in the examples. The centerline of the pipe is at x=0 and the thickness of the mesh is t. The temperature gradient of the mesh is T(x) with the ambient water temperature being represented by a constant, Tₐ. The dotted rectangle is an example infinitesimal mesh section that was used in the integration.

FIG. 12 is a diagram of an infinitely small increment of the mesh for conservation of energy analysis. q′ₓ represents the incoming heat flux from the mesh at x=x and q_{x-dx} is the outgoing heat flux from the mesh at x=x+dx. q′_{c} is the heat flux lost to the contact resistance between the pipe and the mesh.

FIG. 13 is a diagram showing the thermal sensor array of the systems of the examples, and how each sensor can hook up to a sensing bus that was designed by the makers of the thermal sensors, Dallas Semiconductor Corp.

FIG. 14 is a photograph of the structure of the one pipe proof of concept system and the two pipe proof of concept system demonstrated in the examples.

FIG. 15 is a photograph of the structure of the three pipe proof of concept system demonstrated in the examples.

FIG. 16 is a schematic detailing the locations of the five thermal sensors in the agar gel for the experimental setups. One is placed over the pipe, with the other four being placed at quarter-inch intervals away from the pipe.

FIG. 17 is a schematic of a proof of concept apparatus for localized radiative heating of a biofilm support (stainless steel mesh) and biofilm (agar gel). In addition to the thermocouple dots shown, thermocouples were placed in the agar gel to measure the temperature profile as a function of distance from the copper pipe conduits.

FIG. 18 is a plot of the temperature gradient of the one pipe configuration as a function of the distance from the pipe centerline, with the line representing the theoretical equation and the dots indicating the measured experimental temperatures.

FIG. 19 is a plot of the temperature gradient of the two pipe configuration as a function of the distance from the pipe centerline, with the line representing the theoretical equation and the dots indicating the measured experimental temperatures.

FIG. 20 is a plot of the temperature gradient of the three pipe configuration as a function of the distance from the pipe centerline, with the line representing the theoretical equation and the dots indicating the measured experimental temperatures.

### DETAILED DESCRIPTION

The disclosed bioreactor insert apparatus, related bioreactors, and related methods can provide thermal control and/or substance delivery to microorganisms formed on a mesh or membrane support to improve the efficiency and cost of the wastewater treatment process. Wastewater treatment impacts public health and the environment. Further, in view of the large volume of wastewater produced worldwide annually, even a small improvement in efficiency will produce a sizeable impact. Localized heating of biofilms in a bioreactor can substantially reduce energy input for heating microorganisms relative to the status quo. The disclosed bioreactor insert apparatus, related bioreactors, and related methods can provide one or more advantages, for example, decreasing the cost of anaerobic wastewater treatment by heating the emergent biofilms growing on submerged meshes (i.e., dynamic membranes), increasing the activity of the biofilms by controlling the temperature of the biofilms to be an optimum temperature for the microorganisms provided thereon, and/or increasing the activity of the biofilms by controlled, localized, delivery of a substance (e.g., nutrients, substrates, disinfectants) to microorganisms on a biofilm.

Wastewater is any water that has been used in the home, in a business, or in an industrial process. Typically, this wastewater is directed to wastewater treatment facilities to be treated and cycled back into the water supply distribution system. There are two primary categories of wastewater: municipal wastewater which comes from mostly residential areas and uses, and industrial wastewater which comes from industrial processes and energy generation facilities. Municipal wastewater treatment is an essential process that affects all people, as nearly 100 trillion gallons of wastewater are generated each year globally. This is a public health issue, and it can easily be broken down into the triple bottom line as there are numerous social, economic, and environmental contexts that wastewater treatment effects. Currently, 36% of the world's population lives in water-scarce regions. Wastewater treatment has major social implications due to the current clean water inaccessibility that disproportionately affects marginalized and impoverished communities. Current wastewater treatment processes clean wastewater and minimize water pollution, but also contribute to air pollution and use a lot of energy. There is a need to improve this process, as nearly 80% of wastewater produced today is released into the environment without adequate treatment, making this a global issue. This is also an economic issue, as wastewater treatment benefits the economy through reuse in areas such as agriculture and energy generation from its by-products. The by-products, such as nutrients and biogas, can be used as fuel and aid in projects such as composting, allowing for wastewater to produce additional revenues. However, one of the by-products, methane, has the ability to contribute to greenhouse gas emissions if not collected. Effective wastewater treatment is critical to modern society for providing easy to access clean water while protecting public health.

The disclosed bioreactor insert apparatus has a high relative surface area, for example relative to the insert apparatus volume and/or the overall bioreactor reaction volume, which apparatus is capable of generating complex flow patterns and increasing treatment efficiency/biological conversion activity in a biologically-active reactor. The high surface area structure (e.g., a 3D-printed structure) incorporates multiple biofilm support structures (e.g., meshes such as conductive meshes) at inlet and outlet portions of the structure. The biofilm support structures are configured to operate as a biofilm attachment medium such that, during operation of the insert apparatus in combination with a bioreactor, the biofilm support structures and biofilms thereon can increase overall reaction rate of the bioreactor (e.g., increased net biological activity) and/or perform some solid/liquid separation in the treatment of the wastewater or other influent. The disclosed insert apparatus with high relative surface area, when placed into a bioreactor or in fluid communication with a bioreactor, is known as a dynamic membrane bioreactor (DMBR). Such bioreactors are well-suited to handle waste/wastewater streams from municipalities and a variety of industries including agriculture, dairy, food and beverage, and paper.

By promoting biofilm growth on a support or conductive mesh, mass transfer limitations are reduced and microbial interactions between microorganisms are increased, for example syntrophic microorganisms and methanogens. Further, biofilm growth not only increases treatment efficiency but additionally, for anaerobic applications, maximizes methane production in the bulk of the reactor, thereby maximizing overall methane recovery and minimizing permeate dissolved methane concentrations. Namely, methane produced at locally high concentrations (e.g., substantially above equilibrium methane-in-water concentrations for the reactor operating temperature) is provided with substantial residence time in the reactor to equilibrate with and be released into the reactor gas headspace, where the methane can be desirably recovered as a product instead of being lost via the permeate (which is an environmental pollutant in addition to a reduction in product yield). Further still, to boost the activity of the biofilm, the insert apparatus includes conduits for temperature control of the biofilm supports and/or corresponding biofilms to allow the microorganisms to perform at their optimum temperature.

An illustrative embodiment of a bioreactor insert apparatus according to the disclosure is shown in the figures, discussed in the examples, and described in more detail below. The illustrative embodiment includes a 3D-printed structure with (e.g., stainless steel) mesh supports for biofilm formation thereon, which are attached to influent (or recirculating) and permeating (or exit) branches of the biofilm support structure to efficiently treat waste/wastewater streams, and which can aid in biogas production in anaerobic applications. The stainless steel meshes act as a platform for biofilm growth. Additionally, in anaerobic systems, stainless steel or other metallic/electrically conductive materials for the meshes facilitate transfer of electrons between syntrophic and methanogenic microorganisms, resulting in favorable thermodynamics which lead to increased organics removal and methane production. The bulk liquid of the reactor is continuously recirculated so that the wastewater is forced through the biofilm on the influent and permeating branches many times during its residence time in the reactor and so that microorganisms develop a robust biofilm on the influent and permeating meshes. Periodically, the recirculation direction can be reversed so that not all the microorganisms accumulate inside and/or outside of the biofilm structure. The biofilms on the influent and permeating branches generally can have one or both of biological activity for product formation and some degree of solid/liquid separation. The influent branch dynamic membrane biofilms are highly active as they are responsible for the majority of the organics removal, and they provide some barrier to solids transport. Consequently, in anaerobic systems, they will produce the majority of methane via methanogens. The dynamic membrane biofilm that forms on the permeating meshes can be primarily responsible for retaining solids in the reactor and provides additional organics removal, thus providing a more substantial barrier to solids transport and some biological activity for further product formation. This can be useful as it lessens the solids loading in the outlet stream of the insert apparatus, which could be directed to a membrane filtration unit for final permeate clarification. Some attached microbial growth may occur on the 3D-printed structure itself, but due to the designed flow pattern, the majority of biofilm will develop on the meshes. In typical operation, transmembrane pressure (TMP) of the system can be monitored and periodic backwashing can be employed when the TMP reaches a designated threshold, for example at or below 60 kPa to prevent biofilm breakdown (e.g., a threshold TMP of 5, 10, 20, 30, 40, 50, or 60 kPa TMP below which the reactor would normally operate and above which backwashing can be implemented to reduce TMP). Suitably, the reaction system can be normally operated at a TMP in a range of 0.01 kPa to 20 kPa (e.g., at least 0.01, 0.1, or 1 kPa and/or up to 5, 8, 10, 15, or 20 kPa), with periodic backwashing or other cleaning when the TMP reaches or exceeds the designated threshold. By harnessing biofilm treatment on influent (or exit) and permeating (or recirculation) branches, and by utilizing temperature control of the biofilm support and corresponding biofilm, the disclosed bioreactor insert apparatus can produce excellent quality effluent with low capital and operating costs.

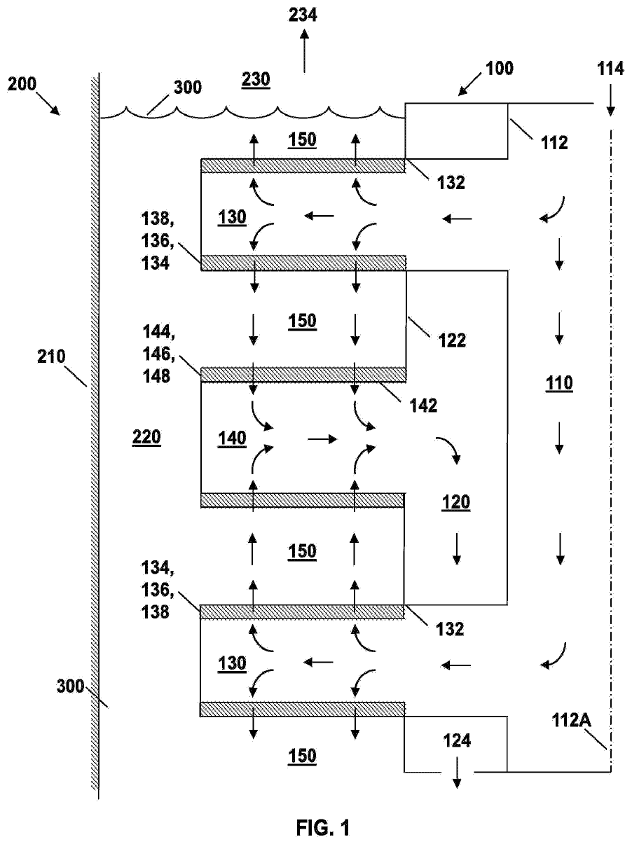

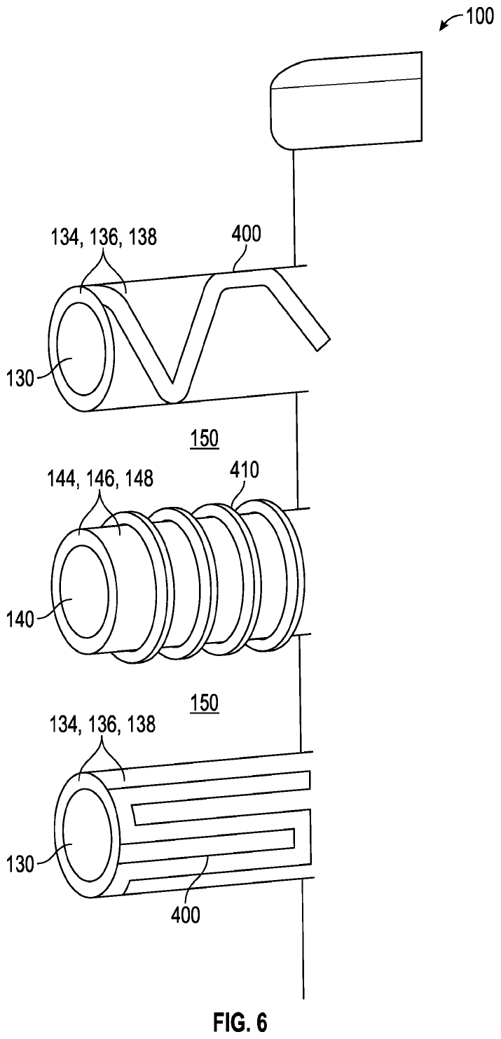

The present disclosure generally relates to a bioreactor insert apparatus 100 and corresponding bioreactor 200, having a structure, for example, as illustrated in FIGS. 1-5. Not illustrated in FIGS. 1-5 are exit conduits and optional recirculation conduits, which are illustrated in FIGS. 6-8. Referring to FIGS. 1-5, the bioreactor insert apparatus 100 provides a means for circulation of reaction medium 300 within the bioreactor 200, a biofilm support, and biological treatment of an inlet feed to the reactor 200/insert apparatus 100.

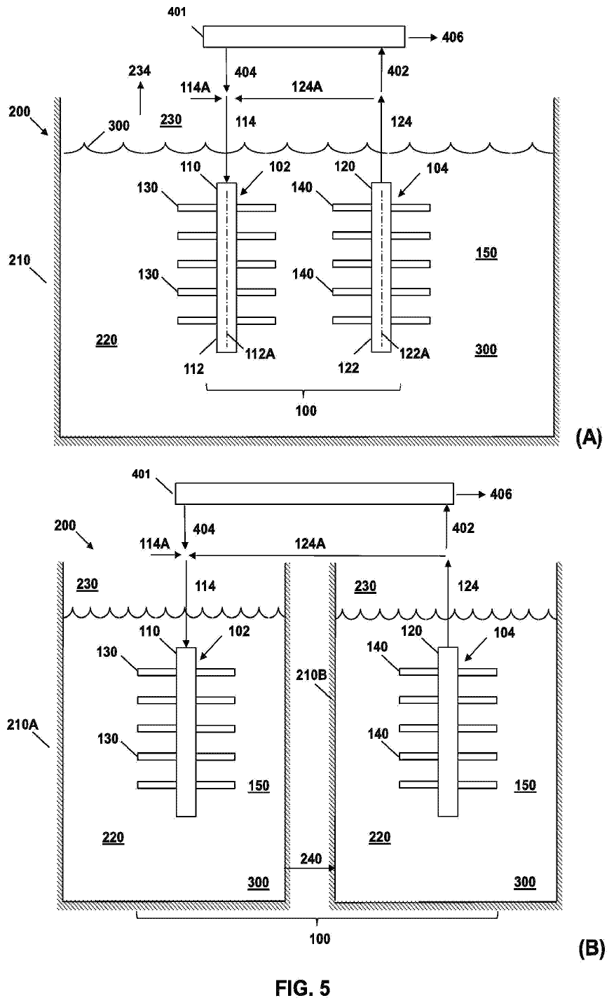

The insert apparatus 100 includes an inlet volume 110 in fluid communication with a first fluid inlet 114 and an outlet volume 120 in fluid communication with a first fluid outlet 124. The first fluid inlet 114 can be an inlet to the bioreactor 200 including the insert apparatus 100 therein, such that feed (e.g., wastewater or otherwise) initially enters the insert apparatus 100 before entering the bioreactor reaction medium 300. The inlet volume 110 can be defined by any suitable geometric structure for liquid flow, for example a cylindrical tube or pipe 112 with centerline 112A as illustrated in FIGS. 1-5. The first fluid outlet 124 likewise can be an outlet to the bioreactor 200 as well as the insert apparatus 100, such that effluent (e.g., remediated wastewater permeate or otherwise) leaving the insert apparatus 100 via the first fluid outlet 124 also exits the bioreactor 200 without recontacting the bioreactor reaction medium 300. The outlet volume 120 can be defined by any suitable geometric structure for liquid flow, for example an annular tube or pipe 122 with centerline 112A as illustrated in FIGS. 1-4 or as a cylindrical tube or pipe 122 with centerline 122A as illustrated in FIG. 5.

The insert apparatus 100 further includes a plurality of exit volumes 130 and a plurality of recirculation volumes 140. Each exit volume 130 can be in direct or indirect fluid communication with the inlet volume 110, and it includes at least one exit biofilm support 134 positioned at a boundary between the exit volume 130 and an external volume 150 outside the bioreactor insert apparatus 100 (e.g., and suitably within the bioreactor 200 and/or reaction medium 300 therein). The external volume 150 generally includes the area outside of the apparatus 100, in particular the area external to the volumes 110, 120, 130, and 140. The exit biofilm support 134 is adapted to promote growth, attachment, and metabolism of microorganisms in the form of a biofilm 136 thereon. The exit biofilm support 134 is adapted to permit fluid and solid transport across the support 134 as well as between the exit volume 130 and the external volume 150, for example based on its partially open structure as defined by a mesh or other suitable structure. When the exit biofilm support 134 further includes the biofilm 136 thereon, fluid (liquid and gas) transport between the volumes 130/150 is still permitted, but solid transport could be still permitted, somewhat impeded, or substantially prevented, depending on the nature of the biofilm 136 and its attachment to the support 134. A relatively thin and/or loosely attached biofilm 136 does not substantially limit solid transport, while a relatively dense and/or strongly attached biofilm 136 can at least partially limit solid transport. The exit volume 130 can be defined by any suitable geometric structure for liquid flow, for example a tube or pipe 132 connected directly to or otherwise in fluid communication with the inlet volume 110 as illustrated in FIGS. 1-5. Each recirculation volume 140 can be in direct or indirect fluid communication with the outlet volume 120, and it includes at least one recirculation biofilm support 144 positioned at a boundary between the recirculation volume 140 and the external volume 150 outside the insert apparatus 100. The recirculation biofilm support 144 is adapted to promote growth, attachment, and metabolism of microorganisms in the form of a biofilm 146 thereon. The recirculation biofilm support 144 is adapted to permit fluid and solid transport across the support 144 as well as between the recirculation volume 140 and the external volume 150, for example based on its partially open structure as defined by a mesh or other suitable structure. When the recirculation biofilm support 144 further includes the biofilm 146 thereon, fluid (liquid and gas) transport between the volumes 140/150 is still permitted, but solid transport could be still permitted, somewhat impeded, or substantially prevented, depending on the nature of the biofilm 146 and its attachment to the support 144 as described above for the support 134/biofilm 136. The recirculation volume 140 can be defined by any suitable geometric structure for liquid flow, for example a tube or pipe 142 connected directly to or otherwise in fluid communication with the outlet volume 110 as illustrated in FIGS. 1-5. The microorganisms or microbes forming the biofilms 136, 146 are not particularly limited and can include bacteria (e.g., anaerobic, anoxic, or aerobic), archaea, or other microorganisms such as algae or other eukarya as well as viruses. Any microbial community that forms a biofilm is suitable.

Referring to FIGS. 6-8, the insert apparatus 100 further includes at least one conduit for example, an exit conduit 400 when mounted adjacent to an exit volume 130 and/or an exit biofilm support 134, or a recirculation conduit 410 when mounted adjacent to a recirculation volume 140 and/or a recirculation biofilm support 144. Each conduit comprises a thermal fluid inlet 420, a thermal fluid outlet 430, and an interior volume 440 (e.g., volume between inlet/outlet and defined by conduit outer wall(s)) adapted to receive and permit flow of a thermal fluid (or working fluid, for example a liquid such as or including water) from the thermal fluid inlet 420 to the thermal fluid outlet 430 via the interior volume 440.

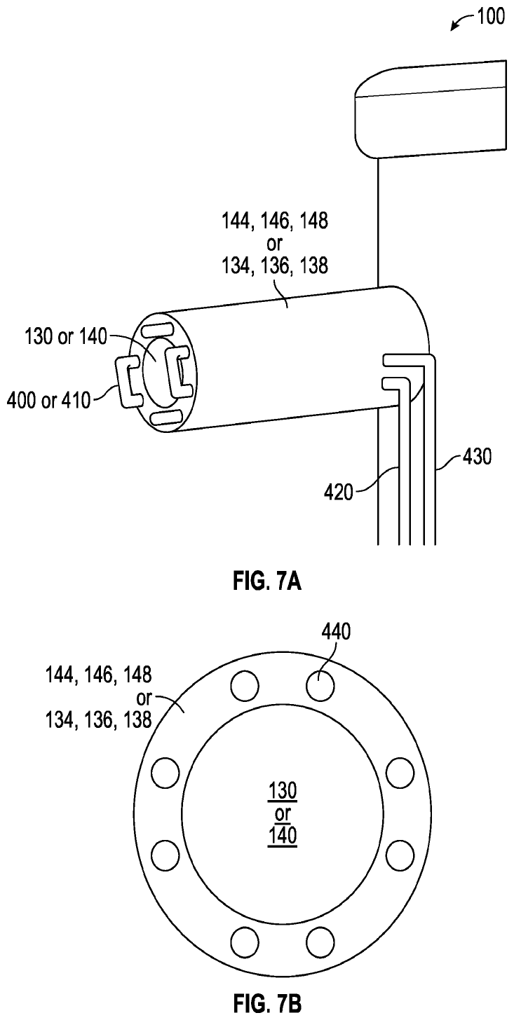

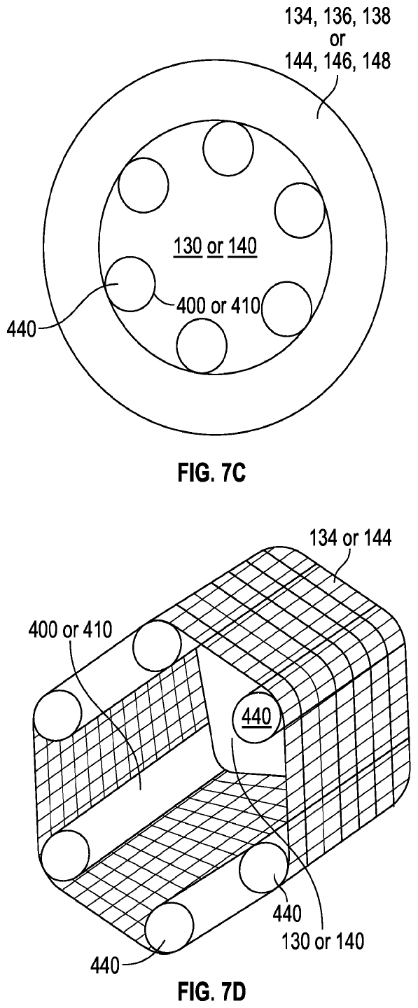

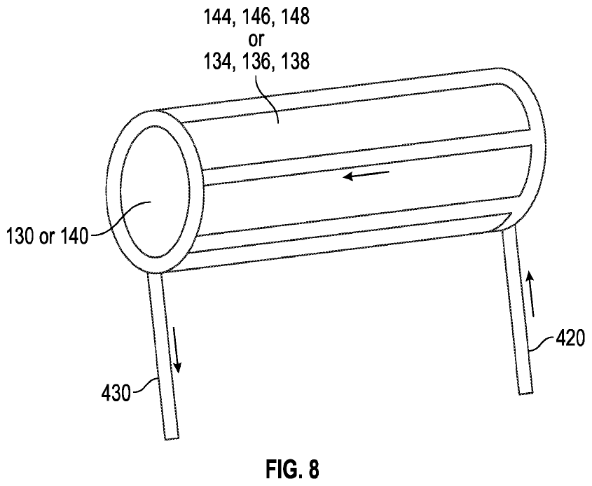

The exit conduit 400 and recirculation conduit 410 can be defined by any suitable geometric structure for liquid flow, for example a pipe, tube or other structure connected directly to or otherwise in fluid communication with the thermal fluid inlet 420 and the thermal fluid outlet 430. The exit conduit 400 and recirculation conduit 410 can be defined by any suitable configuration. FIGS. 6 and 8 illustrate non-limiting configurations for conduits when mounted adjacent to an exit biofilm 136 or biofilm support 134 or recirculation biofilm 146 or biofilm support 144, such as where the conduit “snakes” the length of the exit or recirculation biofilm and/or biofilm support, where the conduit wraps in a spiral around the length of the exit or recirculation biofilm and/or biofilm support, or an “out and back” configuration, where the conduit runs away from the center of the insert apparatus toward the end of the exit or recirculation volume proximal to the external volume outside of the insert apparatus, and then runs back toward the center of the apparatus and so on. As shown in FIGS. 7A and 7B, the conduit can be provided adjacent to an exit biofilm 136 or biofilm support 134 or recirculation biofilm 146 or biofilm support 144 in that the biofilm or biofilm support is provided on two sides of the conduit, such that the conduit is encompassed by the biofilm and/or biofilm support. FIGS. 7C and 7D illustrate yet another configuration, wherein the conduit is provided adjacent to the exit volume 130 or recirculation volume 140 as well as the biofilm and/or biofilm support. The positions of the thermal fluid inlet 420 and thermal fluid outlet 430 are not limiting and non-limiting examples are shown in FIGS. 7 and 8.

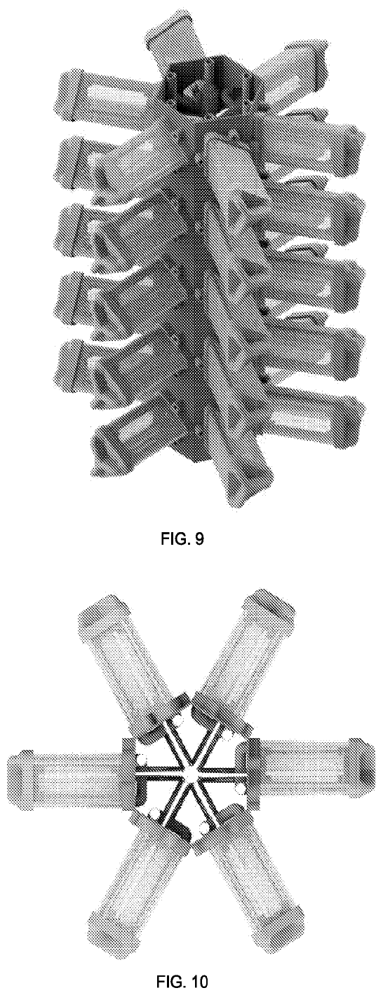

Although the bioreactor insert apparatus 100 is generally illustrated in the figures as a tree-type structure with central flow conduits (e.g., inlet and outlet volumes 110, 120) and a plurality of outwardly directed biofilm support and flow conduits attached thereto (e.g., exit and recirculation volumes 130, 140), the insert apparatus 100 is not limited to the particular illustrated structure. One alternative configuration is illustrated in FIGS. 9 and 10, wherein the treatment fluid flows from the outside of the insert apparatus, through the biofilm/biofilm support, to a central channel of the insert apparatus, and the thermal fluid input and thermal fluid output are run down the center of the apparatus and into each of the three conduits of each branch. The insert apparatus 100 is generally designed to provide a relatively high total surface area (A) of all biofilm supports 134, 144 combined (e.g., on all exit volumes 130 and recirculation volumes 140), relevant because the surface area is the corresponding area for biofilm 136, 146 formation and microorganism metabolism to which the overall reaction rate is proportional. The relative surface area of the insert apparatus 100 can be expressed in a variety of ways. For example, a relative surface area ratio (A/V_{A}) can be defined as the total surface area (A) described above relative to the bioreactor insert apparatus 100 volume (V_{A}), for example including the total volume from the inlet volume 110 (e.g., cylinder), the outlet volume 120 (e.g., annulus), the exit volumes 130 (e.g., exit tubes), and the recirculation volumes 140 (e.g., recirculation tubes). In some embodiments, the ratio (A/V_{A}) can be at least 1 m⁻¹ (or equivalently 1 m²/m³ as a specific surface area parameter). There is no particular upper bound, as it can be desirable to maximize or otherwise increase the ratio (A/V_{A}) with any desired geometry and/or manufacturing technique (e.g., a 3D printing process as illustrated in the examples or otherwise). Taking into account other operational characteristics of the insert apparatus 100 (e.g., pressure drop during operation), the ratio (A/V_{A}) suitably can be in a range from 1 m⁻¹ to 1000 m⁻¹, for example at least 1, 2, 3, 5, 10, 20, or 50 m⁻¹ and/or up to 5, 10, 15, 20, 50, 100, 200, 500, or 1000 m⁻¹. Alternatively or additionally, a relative surface area ratio (A/V_{R}) can be defined as the total surface area (A) described above relative to the bioreactor 200 volume (V_{R}), for example including the volume of the bioreactor 200 reaction vessel 210 or the interior reaction volume 220/reaction medium 300 volume. In some embodiments, the ratio (A/V_{R}) can be at least 0.1 m⁻¹ (or equivalently 0.1 m²/m³ as a specific surface area parameter). As noted above, there is no particular upper bound, as it can be desirable to maximize or otherwise increase the ratio (A/V_{R}). Taking into account other operational characteristics of the insert apparatus 100 (e.g., pressure drop during operation), the ratio (A/V_{R}) suitably can be in a range from 0.1 m⁻¹ to 100 m⁻¹, for example at least 0.1, 0.2, 0.3, 0.5, 1, 2, or 5 m⁻¹ and/or up to 0.5, 1, 1.5, 2, 5, 10, 20, 50, or 100 m⁻¹.

In an embodiment, bioreactor insert apparatus 100 can be formed by a 3D printing or additive manufacturing process. For example, a 3D printing or additive manufacturing process can be used to form the entire insert apparatus 100 or one or more components thereof, such as the inlet and/or outlet structures, 102, 104, the exit and/or recirculation volumes 130, 140, the biofilm supports 134, 144, the conduits 400, 410, the thermal fluid inlet 420, the thermal fluid outlet 430, etc. The specific types of materials used in the 3D printing process and the corresponding insert apparatus 100 structure are not particularly limited, for example including any suitable metal, plastic, or ceramic material amenable to an additive manufacturing process. Example materials include stainless steels, polypropylenes (PP), nylons/polyamides (PA), acrylic resins, and polyethylene terephthalates (PET). The use of 3D printing to form the insert apparatus 100 can provide advantages such as selection of a custom geometric design to improve or control operation of the insert apparatus 100 and corresponding bioreactor 200. The use of 3D printing can also provide modular designs for the insert apparatus 100 such that certain components thereof can be changed or replaced while other components can continue to be used in their current form. Such modular design can allow selective replacement of damaged or worn components, and it can also allow selective replacement of components with different materials or geometries for different operational characteristics of the insert apparatus 100 and bioreactor 200.

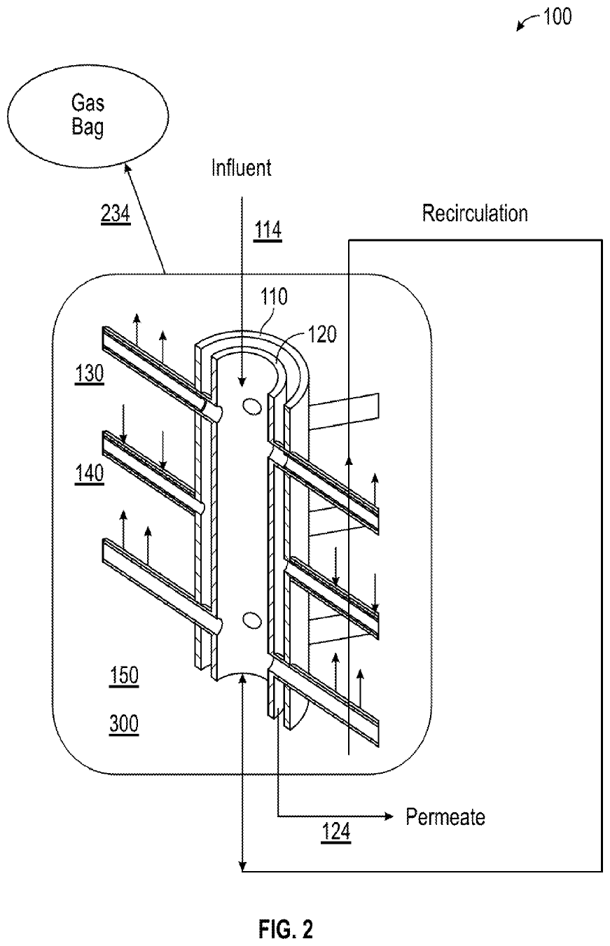

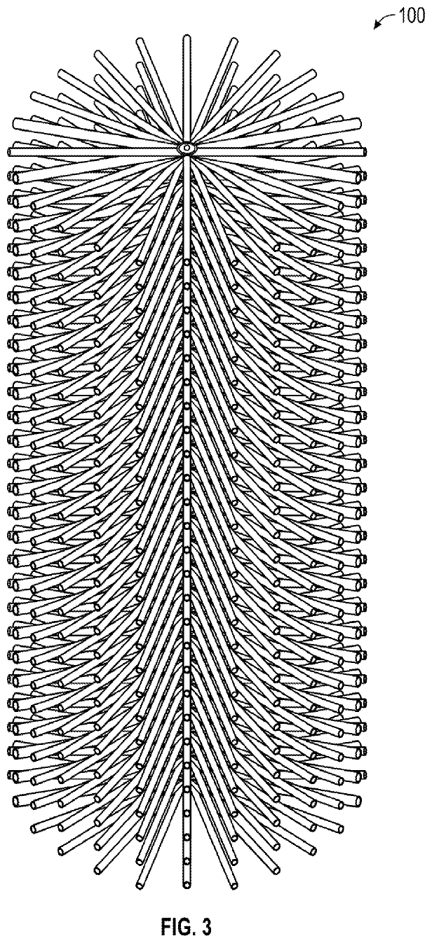

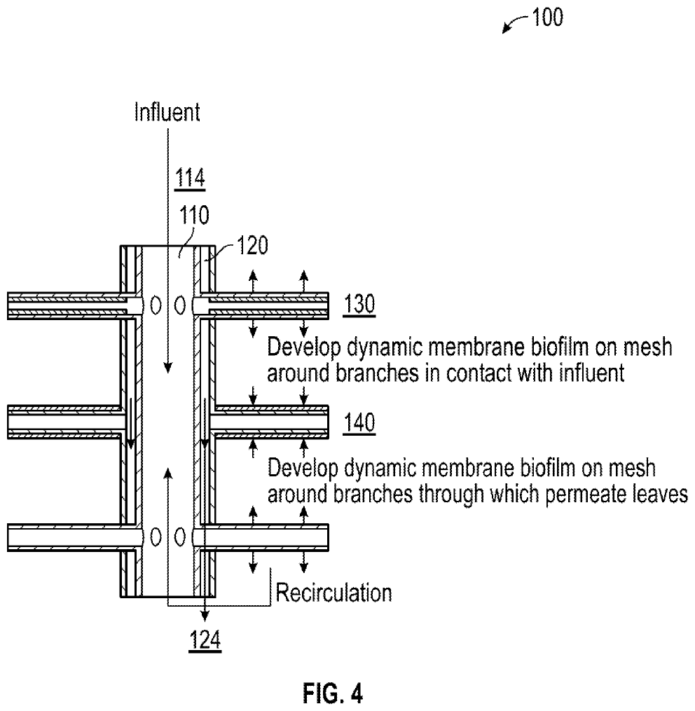

In an embodiment and as generally illustrated in FIGS. 1-4, the bioreactor insert apparatus 100 can be a single (unitary) structure, for example where a single insert apparatus 100 or multiple insert apparatus 100 can be placed within the bioreactor 200 for operation. The single-structure apparatus 100 can include the inlet volume 110, the outlet volume 120 attached or mounted to/around the inlet volume 110 (e.g., as an annular region as illustrated), the plurality of exit volumes 130 attached or mounted to/around the inlet volume 110 and in fluid communication there-with (e.g., as tubes as illustrated), and the plurality of recirculation volumes 140 attached or mounted to/around the outlet volume 120 and in fluid communication therewith (e.g., as tubes as illustrated). In the particularly illustrated embodiment, the inlet volume 110 can include a cylindrical tube 112 in fluid communication with the first fluid inlet 114. The cylindrical tube 112 defines a central axis 112A generally corresponding to the centerline of the cylindrical tube 112. The outlet volume 120 can include an annular tube 122 around the cylindrical tube 112 and in fluid communication with the first fluid outlet 124 (e.g., with the annular tube 122 generally having the same central axis/centerline 112A as the cylindrical tube 112). The exit volumes 130 can include (cylindrical) exit tubes 132 in fluid communication with the cylindrical tube 112 inlet volume 110, for example being mounted to and extending radially outward relative to the central axis 112A of the cylindrical tube 112. The recirculation volumes 140 can include (cylindrical) recirculation tubes 142 in fluid communication with the annular tube outlet volume 120, for example being mounted to the annular tube 122 and extending radially outward relative to the central axis 112A. Although the tubes 132, 142 are illustrated as cylindrical tubes, they can have any desired shape (e.g., tube or duct with a square, rectangular, or other cross section) with the general function of increasing the available biofilm 136, 146 surface area (A) and/or the relative ratios (A/VA) and (A/VR). In representative embodiments, suitable L/D ratios for the tubes 132, 142 can range from 2 to 100 (e.g., at least 2 and/or up to 100). FIG. 3 illustrates an embodiment with relatively long, thin tubes 132, 142 (i.e., relatively higher L/D ratios) for comparatively more relative surface area, for example in relation to the embodiments in FIGS. 2 and 4.

In an embodiment and as generally illustrated in FIG. 5, the bioreactor insert apparatus 100 can include two separate structures, including a first (or inlet/influent/feed) structure 102 and a second (or outlet/effluent/permeate) structure 104. The first structure 102 includes the inlet volume 110 and the exit volumes 130 mounted thereto. The second structure 104 includes the outlet volume 120 and the recirculation volumes 140 mounted thereto. The first and second structures 102, 104 are separate structures with the exit volumes 130 being in fluid communication with the recirculation volumes 140 via the external volume 150, reaction interior volume 220, and/or reaction medium 300. In this embodiment, a single first structure 102 or multiple first structures 102 as well as a single second structure 104 or multiple second structures 104 can be placed within the same bioreactor 200 for operation (e.g., with the same or different number of first and second structures 102, 104 in the bioreactor). In an alternative of this embodiment (not shown), at least one first structure 102 can be placed within a first bioreactor and at least one second structure 104 can be placed within a second bioreactor for operation, for example when the reaction medium from the first reactor is in fluid communication with the reaction medium from the second reactor, in which case reaction medium passing through the first structure 102 and into the first reactor reaction medium can then travel to the second reactor reaction medium and into/through the second structure. The first structure 102 can include the inlet volume 110 and the plurality of exit volumes 130 attached or mounted to/around the inlet volume 110 and in fluid communication therewith (e.g., as tubes as illustrated). The second structure 104 can include the outlet volume 120 and the plurality of recirculation volumes 140 attached or mounted to/around the outlet volume 120 and in fluid communication therewith (e.g., as tubes as illustrated). In the particular illustrated embodiment, the inlet volume 110 can include a cylindrical tube 112 in fluid communication with the first fluid inlet 114. The cylindrical tube 112 defines a central axis 112A generally corresponding to the centerline of the cylindrical tube 112. Likewise, the outlet volume 120 can include a cylindrical tube 122 in fluid communication with the first fluid outlet 124. The cylindrical tube 122 defines a central axis 122A generally corresponding to the centerline of the cylindrical tube 122. The exit volumes 130 can include (cylindrical) exit tubes 132 in fluid communication with the cylindrical tube 112 inlet volume 110, for example being mounted to and extending radially outward relative to the central axis 112A of the cylindrical tube 112. The recirculation volumes 140 can include (cylindrical) recirculation tubes 142 in fluid communication with the cylindrical tube 122 outlet volume 120, for example being mounted to and extending radially outward relative to the central axis 122A of the cylindrical tube 122.

In some embodiments, the first fluid outlet 124 can be in fluid communication with the first fluid inlet 114, for example including piping/tubing connections external to the bioreactor insert apparatus 100 and/or to the bioreactor 200 system more generally. In this way, the apparatus 100 and/or bioreactor 200 can include an external recycle of effluent 124A that has gone through one pass of the insert apparatus 100 such that a portion of the effluent can be recycled for further passes in the insert apparatus 100 if desired, thus being mixed with feed prior to being introduced via the first fluid inlet 114. Likewise, a portion of the effluent can be withdrawn as a final product stream 402 or 406, for example being passed through an external membrane filter 400 for final clarification.

The bioreactor insert apparatus 100 includes a plurality of exit volumes 130 and a plurality of recirculation volumes 140, the specific number of which is not particularly limited, and can be the same or different as between the exit volumes 130 and the recirculation volumes 140. For example, the number of exit volumes 130 can be at least 10, 20, 30, 40, 50, 60, 80, 100, 150, or 200 and/or up to 20, 40, 60, 80, 100, 120, 160, 200, 300, 400, 500, or 1000. The foregoing ranges generally correspond to the number of exit volumes 130 for a single apparatus 100 or a single inlet volume 110 (e.g., cylindrical tube 112 as illustrated). Generally, a larger number of exit volumes 130 increases the biofilm support 134/biofilm 136 surface area (A) relative to the volume of the apparatus 100 (VA), thus also increasing the specific biological activity of the apparatus 100 but also potentially increasing the pressure drop across the apparatus 100. The number of recirculation volumes 140 can be generally in the same ranges as for the exit volumes 130. In various embodiments, there can be more or fewer recirculation volumes 140 as compared to exit volumes 130. For example, the number of recirculation volumes 140 relative to the number of exit volumes 130 can be at least 0.2, 0.4, 0.6, 0.8, 0.9, or 1.0 and/or up to 1, 1.2, 1.5, 2, 3, 4, or 5. Generally, a larger number of recirculation volumes 140 increases the biofilm support 144/biofilm 146 surface area (A) relative to the volume of the apparatus (VA), thus increasing the specific biological activity of the apparatus 100 but also potentially increasing the pressure drop across the apparatus 100.

In an embodiment, the biofilm supports 134 and/or 144 can be in the form of a mesh, for example a grid structure with substantial open areas between grid wires or elements. The biofilm supports 134 and/or 144 more generally can be any solid support structure, preferably one with periodic or otherwise distributed holes or orifices therein to promote liquid-biofilm contact and flow between exit volumes 130, recirculation volumes 140, and the external volume 150 of liquid and solids, such as suspended anaerobic, anoxic, and/or aerobic microorganisms and/or suspended solids from a wastewater influent feed to the apparatus 100 and/or the bioreactor 200. The biofilm supports 134, 144 provide some flow resistance, in particular when biofilms 136, 146 are on the supports 134, 144. The flow resistance limits (but does not prevent) mixing between adjacent compartments, which in turn allows metabolic activity by microorganisms in the biofilm 136 as inlet fluid passes from the inlet volume 110, into an individual exit volume 130, through the biofilm support 134 with biofilm 136 thereon, and into the external volume 150 corresponding to the bulk reaction medium 300. Depending on the coarseness/fineness of the biofilm support 134 and the density/thickness of the biofilm 136 thereon, some, all, or none of the particulate solid material in the inlet feed can be retained by or transmitted through the biofilm support 134/biofilm 136 from the exit volume 130 to the external volume 150. Likewise, there can be metabolic activity by microorganisms in the biofilm 146 as fluid passes from the bulk reaction medium 300, through the biofilm support 144 with biofilm 146 thereon, into an individual recirculation volume 140, and into the outlet volume 120. Similarly, depending on the coarseness/fineness of the biofilm support 144 and the density/thickness of the biofilm 146 thereon, some, all, or none of the particulate solid material in the bulk reaction medium 300 can be retained by or transmitted through the biofilm support 144/biofilm 146 from the external volume 150 to the recirculation volume 140. The biofilms 136, 146 generally transmit and/or generate influent organic (reactant) species, intermediate metabolic products (e.g., organic acids), and final metabolic products (e.g., methane). The biofilm supports 134, 144 as a mesh or other structure with open areas can have mesh spacings/open areas on the micron scale, for example on the order of 100 microns, 101 microns, or 102 microns. Standard mesh designations such as 20 mesh (841 microns) to 400 mesh (37 microns) can be used, as well as smaller or other non-standard mesh sizes. More generally, suitable mesh spacings/open areas can be in a range from 1 micron to 1000 microns, for example at least 1, 2, 5, 10, 20, 50, 100, or 200 microns and/or up to 10, 20, 50, 100, 200, 500, 700, 900, or 1000 microns. In an embodiment, the biofilm support can further include a thermocouple adapted to measure and store or transmit temperature data to a receiver to allow temperature adjustments during the operation of the bioreactor.

In an embodiment, the biofilm supports 134 and/or 144 can include an electrically conductive material, for example being formed from a metallic material, a conductive carbon material, or otherwise electrically conductive material, such as a metallic mesh or a high-surface area conductive carbon mesh. The electrically conductive biofilm supports 134, 144 permit electron transport within the supports 134, 144, but the supports 134, 144 are not necessarily connected to an external electrical power supply or voltage source, for example being electrically insulated relative to the reaction vessel (wall) and other biofilm supports. The electrically conductive biofilm supports 134, 144 allow transport of electrons between biofilm microorganisms thereon, some of which can generate electrons during metabolic (oxidation) processes, and some of which can consume electrons during metabolic (reduction) processes, thereby promoting syntrophic metabolic pathways between biofilm microorganisms. In a further embodiment, the biofilm supports 134, 144 can include an electrically conductive carbon cloth mesh (e.g., activated carbon cloth mesh with high specific surface area), for example on a stainless steel or other metallic/electrically conductive support structure.

In an alternative embodiment, the biofilm supports 134 and/or 144 can include an electrically resistive material, for example being formed from an electrically resistive or insulating material such as various plastic materials, natural or synthetic rubbers, or silicon/silicon-based materials. One or more components of the insert apparatus 100 similarly can include an electrically resistive material, for example the inlet, outlet, exit, and/or recirculation volumes 110, 120, 130, 140. Examples of suitable electrically resistive or insulating plastic materials include polyethylene terephthalates (PET), polybutylene terephthalates (PBT), polyether sulfones (PES), polyether ether ketones (PEEK), polypropylenes (PP), polyvinyl chlorides (PVC), etc. The specific resistance or electrical resistivity of the electrically resistive or insulating materials is not particularly limited, but it is suitably at least about 95, 100, 102, 106 or 1010 ohm·m and/or up to about 102, 1010, 1016 or 1026 ohm·m. The electrically resistive material is adapted to heat under electrical current, for example as applied by an external source or other means. For example, an external power source can be electrically connected to the biofilm supports 134, 144, and/or other insert apparatus 100 component (e.g., in single or separate electrical circuits) to produce an electrical current through the supports, which current in turn induces electrical resistance heating in the supports specifically and the reactor environment more generally (e.g., due to heat convection and conduction away from the supports). In some embodiments, the source of current in the supports can be one or more solar panels in electrical connection with the supports, or the source of current can be derived from microorganisms growing in the reactor (e.g., with such microorganisms as could be used in a microbial fuel cell). The heating effect from the electrically resistive supports can be such that the bioreactor can be operated at a mesophilic temperature (e.g., 20° C. to 40° C. or 30° C. to 40° C.) or at a thermophilic temperature (e.g., 40° C. to 60° C.), for example with or without another heating source such as a heating jacket for the reactor, etc.

The bioreactor insert apparatus 100 includes at least one exit conduit 400 mounted adjacent to an exit biofilm support. The bioreactor insert apparatus 100 can also include at least one recirculation conduit 410 mounted adjacent to a recirculation biofilm support. The features described herein related to conduits can apply analogously to exit conduits and recirculation conduits (or other temperature-control/substance-delivery conduits) when present. A recirculation conduit differs from an exit conduit in that a recirculation conduit is mounted adjacent to a recirculation volume and an exit conduit is mounted adjacent to an exit volume. The conduit can include a pipe, tube or other structure for flow of a thermal or other working fluid/liquid for temperature control and/or substance delivery. The adjacent spatial relationship between the conduit and the biofilm support (or biofilm) can include arrangements in which the conduit is on or in direct physical contact with the biofilm support and/or biofilm. Alternatively, the conduit can be substantially close to the biofilm support (e.g., within 1, 2, 4, 6, 8, or 10 mm), for example spaced apart from the biofilm support such that growth of the biofilm thereon would provide contact or closer proximity of the biofilm and the conduit. Proximity between the conduit and the biofilm support or biofilm (if not direct contact) is preferably close enough to permit sufficient conductive heat transfer to maintain the biofilm support and/or biofilm at a desired temperature as described below for different microorganisms or other operating targets.

In an embodiment, the bioreactor insert apparatus further comprises: an outlet volume in fluid communication with a first fluid outlet; at least one recirculation volume (e.g., a plurality of recirculation volumes), each recirculation volume being in fluid communication with the outlet volume and comprising at least one recirculation biofilm support disposed at a boundary between the recirculation volume and the external volume outside the bioreactor insert apparatus, wherein: the recirculation biofilm support is adapted to promote growth, attachment, and metabolism of microorganisms in the form of a biofilm thereon, and the recirculation biofilm support is adapted to permit fluid and solid transport across the recirculation biofilm support and between the recirculation volume and the external volume; and optionally at least one recirculation conduit mounted adjacent to the recirculation biofilm support. Each recirculation conduit comprises a thermal fluid inlet, a thermal fluid outlet, and an interior volume adapted to receive and permit flow of a thermal fluid from the thermal fluid inlet to the thermal fluid outlet via the interior volume; the recirculation conduit interior volume is substantially liquidly isolated from the first fluid inlet, the first fluid outlet, the inlet volume, the outlet volume, the exit volume, the recirculation volume, and the external volume; and the recirculation conduit is adapted to provide heat transfer between a thermal fluid in the recirculation conduit and the recirculation biofilm support, thereby controlling a temperature of the recirculation biofilm support.

Each conduit 400, 410 comprises a thermal fluid inlet 420, a thermal fluid outlet 430, and an interior volume 430. The conduit interior volume is adapted to receive and permit flow of a thermal fluid from the thermal fluid inlet to the thermal fluid outlet via the interior volume. The conduit is substantially liquidly isolated from the first fluid inlet, the inlet volume, the exit volume, and the external volume (e.g., and first fluid outlet, the outlet volume, and the recirculation volume when present). Substantial liquid isolation includes limiting or preventing liquid flow, such as less than 0.01, 0.1, 1, 2, or 5% of liquid volumetric or mass flow rate through the conduit can pass through the conduit wall (e.g., a semi-permeable portion thereof) into the external environment. When the conduit includes a semi-permeable portion, gases, dissolved or ionic species, and/or small particulate solids (e.g., micro- or nano-scale) can pass from the conduit interior volume into the external environment, such as from the thermal fluid to the bulk reaction medium. When the conduit does not include a semi-permeable portion, the conduit interior volume can be completely isolated from the first fluid inlet, the inlet volume, the exit volume, and the external volume (e.g., and first fluid outlet, the outlet volume, and the recirculation volume when present), such that there is essentially no liquid, gas, or solid material passing from the thermal fluid to the bulk reaction medium. The conduit is adapted to provide heat transfer between a thermal fluid in the conduit and the biofilm support (e.g., and any biofilm thereon), thereby controlling a temperature of the biofilm support (e.g., and any biofilm thereon). In particular, controlling or selecting the temperature of the thermal fluid delivered to the conduit in turn permits control of the resulting temperature of the biofilm support or biofilm. Such heat transfer primarily includes conductive transport and optionally includes some convective transport, such as in reaction medium liquid passing through the biofilm and/or flow of reaction medium liquid in the vicinity of the biofilm/biofilm support; these transport effects can alternatively be collectively referenced as radiative heat transport. More specifically, heat transfer and temperature control can include a combination of heat conduction within the solid mesh or other biofilm support material as well as a conductive (and possibly convective) heat loss from the mesh or biofilm support into the bulk liquid reaction medium or external environment.

In embodiments, the exit conduit interior volume is substantially liquidly isolated from the first fluid inlet, the inlet volume, the exit volume, and the external volume and the exit conduit is adapted to provide heat transfer between a thermal fluid in the exit conduit and the exit biofilm support, thereby controlling a temperature of the exit biofilm support. In embodiments, the insert apparatus further comprises at least one recirculation volume comprising a recirculation biofilm support, and optionally at least one recirculation conduit mounted adjacent to the recirculation biofilm support, wherein the recirculation conduit interior volume is substantially liquidly isolated from the first fluid inlet, the first fluid outlet, the inlet volume, the outlet volume, the exit volume, the recirculation volume, and the external volume, and the recirculation conduit is adapted to provide heat transfer between a thermal fluid in the recirculation conduit and the recirculation biofilm support, thereby controlling a temperature of the recirculation support.

In general, the conduits of the disclosure can be formed from any suitable thermally conductive material to promote heat transfer between the thermal fluid and the biofilm support and/or biofilm through the conduit wall. In an embodiment, the exit conduit comprises a metal material (e.g., formed from a metal such as steel, stainless steel, copper or other metal tubing). Conduit materials such as stainless steel can also be selected based on their ability to resist corrosion or other degradation due to exposure to the bulk bioreactor liquid medium. The conduit can be formed from such thermally conductive materials in regions where the conduit is in contact with/adjacent to the biofilm/biofilm support, for example where the conduit also has regions where it is formed from thermally resistive materials to limit heat losses while the thermal fluid is transferred to/from the thermally conductive regions (e.g., to/from an external heater/cooler/pump for recirculation). For example, typical thermal conductivity values (e.g., at a reference temperature of 25° C.) for metals or other thermally conductive materials can range from 5 to 500 W/(m K), for example at least 5, 15, 30, 50, 100, 200, or 300 W/(m K) and/or up to 50, 100, 200, 300, 400, or 500 W/(m K).

In an embodiment, the exit conduit and/or recirculation conduit comprises a thermally insulating material for one or more regions of the conduit that are not adjacent to (e.g., not in contact with) the biofilm/biofilm support. As noted above, the conduit can include thermally resistive tubing/piping etc. attached to the thermally conductive regions in contact with the biofilm/biofilm support. In addition to using a non-metal plastic or other thermally resistive tubing for recirculation in non-heat conducting areas, the conduit can also incorporate a thermal insulation material as a partial sleeve or coating, etc. around a portion of the metal or other thermally conductive conduit material that is not in contact with or otherwise adjacent to the biofilm/biofilm support. For example, 75% (or other selected fraction) of the circumference of the thermally conductive conduit that is generally in contact with the bulk liquid medium can be covered, coated, etc. with an insulating material to limit heat losses into the bulk liquid medium, while the remaining 25% (or other selected fraction) of the circumference of the thermally conductive conduit that is in contact with or directly adjacent (e.g., facing) the biofilm support or biofilm is bare to promote heat conduction at a metal-metal or other highly heat conductive interface. For example, typical thermal conductivity values (e.g., at a reference temperature of 25° C.) for plastics, rubbers, or other thermally resistive materials (e.g., for thermally resistive tubing or piping, thermally insulating sleeve or coating) can range from 0.001 to 5 W/(m K), for example at least 0.001, 0.01, 0.1, 0.2 or 0.5 W/(m K) and/or up to 0.1, 0.4, 0.7, 1, 2, 3, 4, or 5 W/(m K).

In an embodiment, the exit conduit and/or recirculation conduit comprises a semi-permeable material adapted to permit selective transport of a (non-liquid) substance in the thermal fluid through the semi-permeable material, for example into or in the immediate vicinity of the biofilm support or biofilm such that the transported substance can then promote, stimulate, regulate, or otherwise control growth of microorganisms in the biofilm. Such transport and delivery of the substance from the thermal fluid and through the semi-permeable material can be into one or more regions of the exit volume, the recirculation volume, the external volume, etc. that are sufficiently close to the target biofilm support or biofilm. The non-liquid substances for selective transport can include one or more of gases, dissolved or ionic species, and/or small particulate solids (e.g., micro- or nano-scale solids). Specific examples of substances for delivery include oxygen gas (e.g., with nitrogen gas as in air), trace metals, nitrogen or nitrogen-containing compounds (e.g., as a nutrient), phosphorous or phosphorous-containing compounds (e.g., as a nutrient), nanoparticles, bleach, sodium hydroxide, nutrients more generally, etc. Semi-permeable materials (e.g., semi-permeable membranes) are generally known in the art and are commercially available for a variety selective transport target substances. For example, a commercially available OXYMEM membrane (available from DuPont) can be used to selectively transport and supply oxygen gas from air to a biofilm. In an embodiment, the thermal fluid comprises a substance (e.g., gas, dissolved species, small particulate solid in addition to the transporting liquid medium) for delivery to the biofilm; at least one conduit comprises a semi-permeable material adapted to permit selective transport of the substance from the thermal fluid through the semi-permeable material; and the substance is delivered (or transported) to the biofilm through the semi-permeable material.

The semi-permeable material can be incorporated into the conduit in a variety of configurations. In some embodiments, the semi-permeable material can be formed from a thermally conductive material through which heat is conducted for temperature control. In some embodiments, the semi-permeable material can be in a separate region from that where heat transfer is occurring (e.g., a separate semi-permeable tubing connected to the copper or other heat conductive tubing). In some embodiments, a wall portion of a copper, metal, or other heat conductive conduit can be replaced with a semi-permeable material as a liquid-impermeable barrier that still permits selective substance transport and delivery. The semi-permeable material is generally positioned so that it delivers nutrients or other selectively transported substance spatially close to the biofilm support/biofilm.

In an embodiment, the exit conduit and/or recirculation conduit is in fluid communication with one or more of a heater, cooler, and pump for circulation of a temperature-controlled thermal fluid through the conduit. For example, the conduit can be in fluid communication with the heater and the cooler; and the conduit can be adapted to cycle between heating and cooling states in which relatively warmer and cooler thermal fluids are transported through the conduit to expand and contract the biofilm support and any biofilm thereon (e.g., as part of a cleaning or other maintenance process for the bioreactor). Optionally, the heater and/or cooler can be powered by a power supply connected to a PID controller to allow for the precise control over the temperature of the thermal fluid.

In an embodiment, the exit conduit and/or recirculation conduit is in fluid communication with a reservoir containing the thermal fluid therein. For example, the reservoir can include heating and/or cooling elements such that the reservoir also serves as the heater and/or cooler components described above. The reservoir can be fluidly connected to the pump for circulation. The thermal fluid can be circulated through the conduit at any suitable velocity. For example, the thermal fluid flow velocity should be fast enough that there is little variation between the influent and effluent temperatures, but not so fast as to use excessive amounts of energy to pump the working fluid through the system. In various embodiments, the thermal fluid (e.g., in the reservoir or otherwise) can include one or more liquids selected for their heat transport properties, for example including water, glycols (e.g., ethylene glycol), oils, etc. In embodiments, the thermal fluid comprises water. In addition to the liquid providing the heat for temperature control, the thermal fluid additionally can contain any of the substances described above for selective transport and delivery to the biofilm.

In an embodiment, the conduit is adapted to maintain the biofilm support (e.g., and any biofilm thereon) at a temperature in a range of 25° C. to 40° C. More generally, suitable target temperatures can include those appropriate for mesophilic microorganisms in the biofilm, for example at least 25, 30, 32, or 35° C., up to 30, 35, 37, or 40° C., and/or not higher than 36° C., 37° C., 38° C., 39° C., or 40° C.

In an embodiment, the conduit is adapted to maintain the biofilm support (e.g., and any biofilm thereon) at a temperature in a range of 40° C. to 60° C. More generally, suitable target temperatures can include those appropriate for thermophilic microorganisms in the biofilm, for example at least 40, 45, 50, or 55° C., up to 45, 50, 55, or 60° C., and/or not higher than 56° C., 57° C., 58° C., 59° C., or 60° C.

In an embodiment, the conduit is adapted to maintain the biofilm support (e.g., and any biofilm thereon) at a temperature in a range of 60° C. to 100° C. More generally, suitable target temperatures can include those appropriate for hyperthermophilic microorganisms in the biofilm, for example at least 60, 65, 70, or 80° C., up to 70, 80, 90, or 100° C., and/or not higher than 80° C., 90° C., 95° C., 98° C., or 100° C.

In an embodiment, the conduit is adapted to maintain the biofilm support (e.g., and any biofilm thereon) at a temperature of at least 5° C. higher than a bulk liquid temperature in the exit volume or recirculation volume, and in the external volume. More generally, suitable target temperatures can be at least 5, 7, 10, 20, 30, 40, 50, or 60° C. higher and/or up to 10, 12, 15, 20, 30, 40, 50, 60, 80, or 95° C. higher than the bulk liquid temperature to promote one or more of mesophilic, thermophilic, or hyper-thermophilic microorganism growth.

In an embodiment, the exit conduit is adapted to maintain the biofilm support (e.g., and any biofilm thereon) at a temperature in a range of 1° C. to 20° C. More generally, suitable target temperatures can include those appropriate for psychrophilic microorganisms in the biofilm, for example at least 1, 2, 5, 7, or 10° C., and/or up to 6, 8, 10, 12, 15, or 20° C.

In an embodiment, the conduit is adapted to maintain the biofilm support (e.g., and any biofilm thereon) at a temperature of at least 5° C. lower than a bulk liquid temperature in the exit volume, recirculation volume, and in the external volume. More generally, suitable target temperatures can be at least 5, 7, 10, or 12° C. lower and/or up to 8, 10, 12, 15, or 20° C. lower than the bulk liquid temperature to promote psychrophilic microorganism growth.

In an embodiment, the conduit is adapted to maintain the biofilm support (e.g., and any biofilm thereon) at a temperature in a range of 15° C. to 30° C. More generally, suitable target temperatures can be at least 15, 20, or 25° C., and/or up to 20, 25, or 30° C.

In an embodiment, the conduit is adapted to maintain the biofilm support (e.g., a portion of or substantially the entire biofilm support and any biofilm thereon) at a temperature within ±6° C. of a selected setpoint temperature. Typical temperature variation windows accounting for lateral and longitudinal variations in heat transfer within the biofilm support and/or biofilm can be ±0.5, 1, 1.5, 2, 2.5, 3, 3.5, 4, 5, or 6° C. of a selected setpoint temperature. Suitable setpoint temperatures can be selected based on the psychrophilic, mesophilic thermophilic, or hyperthermophilic character of the desired microorganisms in the biofilm, for example including setpoints or 4, 6, 8, 10, 12, or 14° C., for psychrophilic microorganisms; 29, 31, 33, 35, 37, or 39° C., for mesophilic microorganisms; 44, 48, 52, 56, or 58° C., for thermophilic microorganisms; or 64, 68, 74, 80, 86, 92, or 98° C., for hyperthermophilic microorganisms.

In an embodiment, the spatial temperature distribution of the biofilm support (e.g., and any biofilm thereon) can be suitably narrow such that the substantial portion of the biofilm experiences a consistent desired temperature that maximizes its growth while avoiding extreme temperatures that can kill the microorganisms and/or result in lower growth rates. The foregoing temperature ranges, windows, and setpoints can apply to a portion of or substantially the entire biofilm support and any biofilm thereon, such as at least 30, 50, 70, 80, 90, 95, 98, or 99% and/or up to 60, 75, 85, 95, 98, 99, or 100% of the biofilm support/biofilm volume or surface area has the desired temperature range, window, and/or setpoint. The spatial temperature distribution can be determined using a series of spatially distributed thermocouples or other temperature measurement apparatus, computational heat transfer modeling, etc. The threshold of biofilm support/biofilm area or volume accounts for possible small peripheral areas that might not achieve the desired temperature, but the desired temperature facilitating robust microorganism growth can be obtained over a substantial fraction of the biofilm support/biofilm.

In an embodiment, the exit conduit and/or recirculation conduit is adapted to maintain a temperature gradient in the biofilm support (e.g., and any biofilm thereon) with at least two different setpoint temperatures that are at least 2° C. different from each other. For example, the different setpoint temperatures that are at least 2, 3, 5, 7, 10, 15, 20, or 25° C., and/or up to 4, 6, 8, 10, 12, 15, 20, 30, or 40° C. different from each other. The different setpoint temperatures can correspond to two or more conduits or segments thereof in contact with biofilm support or biofilm, which conduits or segments transport thermal fluids having similar temperature differences as described for the setpoints.

In an embodiment, each biofilm support is adjacent to (e.g., in direct contact with) a plurality of conduit segments at spaced-apart locations of the biofilm support. This structure can minimize/reduce lateral and/or longitudinal temperature gradients in the biofilm support and corresponding biofilm by including multiple conduit segments at multiple different lateral and/or longitudinal locations of the biofilm support, for example where the different conduit segments contain thermal fluids at substantially the same temperature. In other cases, this structure can induce or control desired lateral and/or longitudinal temperature gradients in the biofilm support and corresponding biofilm by including multiple conduit segments at multiple different lateral and/or longitudinal locations of the biofilm support, for example where the different conduit segments contain thermal fluids at different temperatures. As shown in FIGS. 6-8, separate conduit segments can include (i) separate conduit tubes in a parallel arrangement from the same or different heating/cooling/pump reservoir(s), and/or (ii) different regions of a single conduit tube that winds/wraps around the biofilm support at different locations. In an embodiment, the plurality of conduit segments can be at evenly spaced-apart locations of the biofilm support. In an embodiment, the plurality of conduit segments can be at unevenly spaced-apart locations of the biofilm support.

In an embodiment, the thermal conductivity along the length of the conduit tube can be varied so that there is more insulation/resistance to heat conduction at the inlet vs. the outlet such that comparatively hotter thermal fluid at the inlet still results in the same or similar biofilm support/biofilm temperature as compared to colder thermal fluid at the outlet (e.g., to reduce longitudinal temperature gradients in the biofilm support/biofilm). For example, a thin intermediate insulating material that reduces in thickness from the inlet to the outlet can be incorporated between the conduit and the biofilm support/biofilm. In other case, the thermal conductivity along the length of the conduit tube can similarly be selected or varied to induce a desired temperature gradient in the biofilm support/biofilm.

In an embodiment, the exit and/or recirculation conduit is positioned at least partially within the exit volume and/or recirculation volume, respectively. This can represent an embodiment in which the conduit is on the inside of the biofilm support or mesh, for example providing structural support for the biofilm support and constituting a part of the exit volume or recirculation volume structure. Non-limiting examples of this configuration are illustrated in FIGS. 7C and 7D. FIG. 7C is a cross-sectional view of an exit volume 130 or recirculation volume 140 comprising a plurality of conduits 400, 410 having interior volumes 440, surrounded by a biofilm 136, 146/biofilm support 134, 144. FIG. 7D is an illustration of an exit volume 130, 140 defined by a plurality of conduits 400, 410 having interior volumes 440 and a mesh biofilm support 134, 144 provided around the conduits.

In an embodiment, the exit conduit and/or recirculation conduit is positioned at least partially within the external volume. This can represent an embodiment in which the conduit is on the outside of the biofilm support or mesh, for example providing structural support for the biofilm support and constituting a part of the exit volume or recirculation volume structure. Non-limiting examples of this configuration are illustrated in FIGS. 6 and 8. FIGS. 6 and 8 are an illustration of an insert apparatus having exit volumes 130 and/or recirculation volumes 140 and conduits 400, 410 positioned external to the biofilm supports 134, 144/biofilms 136, 146 defining the exit volume 130 and recirculation volumes 140 and at least partially within the external volume 150.

In an embodiment, the exit conduit and/or recirculation conduit is positioned at least partially within the exit volume/recirculation conduit and the external volume. This can represent an embodiment in which the conduit is integrally formed with the biofilm support or mesh, for example where smaller lattice or mesh elements span and are affixed to/integral with adjacent conduit tubes, providing structural support for the biofilm support and constituting a part of the exit volume or recirculation volume structure. Non-limiting examples of this configuration are illustrated in FIGS. 7A and 7B. FIG. 7A is an illustration of a conduit integrally formed with the biofilm support or mesh and FIG. 7B is a cross-sectional view of the biofilm 136, 146/biofilm support 134, 144 and the conduit interior volume 440.

In an embodiment, the bioreactor insert apparatus comprises a plurality of the exit volumes, wherein the exit biofilm support is the only fluid communication pathway between the exit volume and the external volume, such that the exit volume is configured to force circulating liquid from the inlet volume, through the exit biofilm support, and into the external volume; and a plurality of the recirculation volumes, wherein the recirculation biofilm support is the only fluid communication pathway between the recirculation volume and the external volume, such that the recirculation volume is configured to receive forced circulating liquid from the external volume, through the recirculation biofilm support, and into the outlet volume.

In an embodiment, the bioreactor 200 can further include attachment or carrier media for microbial growth, for example in the interior reaction volume 220. During operation of the insert apparatus 100 and/or corresponding bioreactor 200, the attachment media can be suspended or otherwise dispersed and circulating in the corresponding aqueous reaction medium 300. The attachment media provide additional surface area within the bioreactor 200 to promote the growth, attachment, and metabolism of microorganisms thereon at locations other than the biofilm supports. The materials for the attachment media are not particularly limited, but they suitably include plastics or polymers, carbon materials, and/or cellulosic materials in particulate form. Suitable materials include plastics such as polyethylenes (PE), high density polyethylenes (HDPE), or polypropylenes (PP), carbon materials such as activated carbon or carbon fibers, and cellulosic materials such as wood chips or fibers. In some embodiments, one or more components or surfaces of the bioreactor 200 and/or apparatus 100 can be formed from similar materials to also promote microorganism growth thereon. The attachment media can have any suitable geometric shape and/or size, for example a geometry that provides sufficient (specific) surface area for microorganism growth and that allows the attachment media to be maintained in suspension during operation of the bioreactor 200. More specifically, the attachment media can be selected so that circulating fluid motion within the bioreactor 200 maintains the attachment media in suspension in the aqueous reaction medium 300 without substantial separation or segregation of the attachment media (e.g., via settling or flotation, depending on the density of the attachment media), For example, the attachment media in particulate form can have a diameter or size in a range from 0.1 μm to 1000 μm, for example at least 0.1, 0.2, 0.5, 1, 2, 5, 10, 20, or 100 μm and/or up to 0.5, 1, 2, 5, 10, 20, 50, 100, 200, or 1000 μm, whether as a size range or average size (e.g., number-, weight-, area-, or volume-based range or average). Alternatively or additionally, the attachment media can have a specific surface area in a range from 1 m⁻¹ to 10,000 m⁻¹ (i.e., m²/m³) for example at least 1, 2, 5, 10, 20, 50, or 100 m⁻¹ and/or up to 10, 20, 50, 100, 200, 500, 1000, 5000, or 10000 m⁻¹, whether as a range or average specific surface area.

In an embodiment, at least 50%, 65%, or 80% of total microorganisms in the bioreactor are incorporated into the biofilms, for example as determined on a weight or number basis. Suitably, the remaining microorganisms are suspended in the bulk reaction medium 300 and/or within the insert apparatus 100, for example with the bulk reaction medium 300 and insert apparatus 100 having generally the same or similar concentrations of suspended microorganisms. Suitably at least 50, 65, 80, 85, 90, 95, or 98% and/or up to 90, 95, 97, 98, 99, or 100% of total microorganisms are incorporated into the biofilms 136, 146 with only a minor amount in the bulk reaction medium 300 and/or insert apparatus 100. Total biological activity in the bioreactor likewise can be at least 80, 85, 90, 95, or 98% and/or up to 90, 95, 97, 98, 99, or 100% attributed to biological activity/conversion at the biofilm 136, 146 surfaces. In some embodiments, the exit supports 134 buildup highly active biofilms 136 and are responsible for a majority of product formation (e.g., methane production in anaerobic configurations). In such cases, the biofilms 146 of the recirculation supports 144 are predominantly useful for solid/liquid separation, limiting solids in the reaction medium 300 from reentering the insert apparatus 100 via the recirculation volumes 140, although potentially still providing some biological activity. In some embodiments, the recirculation supports 144 buildup highly active biofilms 146 and are responsible for a majority of product formation (e.g., methane production in anaerobic configurations). In such cases, the biofilms 136 of the exit supports 134 are predominantly useful for solid/liquid separation, limiting solids in the reaction medium 300 from reentering the insert apparatus 100 via the exit volumes 130, although potentially still providing some biological activity. In some embodiments, the exit supports 134 and the recirculation support 144 buildup highly active biofilms 136, 146, which both contribute to product formation and/or solid/liquid separation.

In an embodiment, the biofilms 136, 146 have methanogenic activity. At least some microorganisms in the biofilm 136, 146 (and bioreactor 200) are methanogens, producing methane from one or more components in the influent feed and/or as produced by other microorganisms as intermediates such as organic acid intermediates. In an embodiment, the biofilms comprising a community of anaerobic microorganisms. In an embodiment, the biofilms comprising a community of aerobic microorganisms. In an embodiment, the biofilms comprising a community of anoxic microorganisms.

In an embodiment, the bioreactor insert apparatus further comprises a biofilm adhered to the biofilm support, the biofilm comprising a community of microorganisms collectively having biological activity. During operation of the insert apparatus 100 and/or corresponding bioreactor 200, the apparatus 100 further includes the biofilms 136, 146 adhered to their respective biofilm supports 134, 144, for example including a biofilm on all exit and recirculation supports 134, 144. Biofilms generally include a group of microorganisms in which distinct cells stick to one another and adhere to a surface. The adherent cells become embedded in a slimy extracellular matrix polymeric substance as a type of hydrogel. Biofilms are useful in industrial settings where beneficial bacterial biofilms can use organic pollutants as an energy source. Digestion of these compounds removes them from a waste stream and releases environmentally benign substances and/or substances useful as reaction product (e.g., methane). The community of microorganisms or microbes forming the biofilms 136, 146 are not particularly limited and can include bacteria (e.g., anaerobic, anoxic, or aerobic), archaea, or other microorganisms such as algae (e.g., for biofuel component production) or other eukarya as well as viruses. Any microbial community that forms a biofilm is suitable. In some embodiments, the biofilm populations can include microbial species collectively having biological activity, for example methanogenic activity. In some embodiments, at least some microorganisms in the biofilms (and reactor) are methanogens, producing methane from one or more components (e.g., organic or other components) in the influent feed and/or as produced by other microorganisms as intermediates such as organic acid intermediates (e.g., volatile organic acids or otherwise). The microorganisms in the biofilm can be selected to have a specific population distribution of different species to provide a desired/tuned composition for digesting waste streams from distinct sources and having a distinct profile of contaminants. The bioreactor 200 can further include suspended microorganisms in the reaction medium 300, which microorganisms can be the same, similar, or different populations of microorganisms (e.g., bacteria, archaea) as in the biofilm with same, similar, or different activities, for example with a different distribution of species types between the free, suspended microorganisms and the adhered, biofilm microorganisms. Likewise, the exit biofilms 136 can have the same, similar, or different community of microorganisms with the same, similar, or different activities as compared to the recirculation biofilms 146. In this way, the biofilms 136, 146 can have the same or different functions, for example with respect to biological activity (e.g., amount or type of products formed) and/or resistance to solids transport.

While the insert apparatus 100 and corresponding bioreactor 200 are particularly illustrated and described in the context of wastewater treatment reactors for methane generation, the apparatus 100 and bioreactor 200 can be used in any context in which the biofilms have corresponding biological activity to convert one or more reaction system reactants or intermediates to one or more desired biological intermediates or products therefrom. Representative examples broadly include fermentation, biopharmaceutical production, assembly of biomolecules, etc. Industrial or other large-scale fermentation is a particularly suitable application for the insert apparatus 100, in particular for open fermentation applications which utilize mixed microbial communities. In various fermentation processes, singular or groups of microorganisms are used in fermenters or bioreactors to produce a desired product. In such applications, the insert apparatus 100 can be used as a bioreactor component for increasing substrate/microorganism contact and organizing microbial communities on its meshes or supports, thereby improving product yields. Examples of suitable types of fermenters or bioreactors include continuously stirred tank reactors (CSTRs), membrane bioreactors (MBRs, such as for ethanol and organic acid fermentation), microcarrier bioreactors, and fluidized bed bioreactors. Examples of suitable fermentation products include those with medical uses (e.g., thienamycin or other antibiotics), industrial or commodity chemicals (e.g., succinic acid or other organic acids; ethanol, propanol, butanol or other alcohols), alcoholic beverages (e.g., ethanol as a product itself or a component of a beer, liquor, or wine beverage), and food and feed products (e.g., glucoamylase or other digestive enzymes). In each of the foregoing fermentation settings, suitable singular or groups of microorganisms are generally known in the art and may be grown/deposited on the supports 134, 144 to form corresponding biofilms 136, 146.

In an embodiment, the insert apparatus 100 can further include a biofilm seed 138, 148 or precursor adhered to the biofilm supports 134 and/or 144, respectively. For example, the biofilm seed 138, 148 can be on some or all exit supports 134 and recirculation supports 144, which can be the same or different seed for the different supports. The biofilm seed 138, 148 can be provided already on the supports 134, 144 prior to operation of the corresponding bioreactor 200 so that start-up times required to establish a newly operational insert apparatus 100 and bioreactor 200 and with a desired biofilm population are reduced. The biofilm seed can comprise a water-soluble adhesive matrix and a community of microorganisms as a biofilm precursor distributed throughout the matrix. For example, the supports 134, 144 can be inoculated or otherwise coated with a population of (live or dormant) microorganisms, and then the microorganisms can be coated, covered, or otherwise adhered to the supports 134, 144. Suitable coatings include water-soluble binders or adhesives (e.g., a water-soluble polymer such as a water-soluble epoxy or polyvinyl alcohol) applied as a matrix (e.g., an air-tight matrix) on the supports 134, 144 to hold the microorganisms in place in the matrix until startup of the insert apparatus 100 and bioreactor 200. The microorganisms are suitably dormant while fixed in the matrix in their seed or precursor form (e.g., as a result of not being fed with substrate while therein). When the insert apparatus 100 is flooded with water, for example an aqueous reactor feed (e.g., wastewater or otherwise) and/or an aqueous microorganism nutrient medium, the water-soluble matrix is removed, exposing and activating the supported microorganisms, thus allowing them to grow and attach to the supports 134, 144 as corresponding biofilms 136, 146. This activation and startup process can be prior to or concurrent with use of the insert apparatus 100 and bioreactor 200 in normal production with circulation through the apparatus.

The bioreactor 200 generally includes a reaction vessel 210 defining an interior reaction volume 220 therein and a bioreactor insert apparatus of the disclosure mounted within the reaction vessel, wherein the external volume corresponds to a portion of the interior reaction volume outside the bioreactor insert apparatus. The reaction vessel 210 can be open or closed to the external environment, but suitably is closed when a gaseous reaction bioproduct (e.g., methane) is recovered from the bioreactor 200. The reaction volume 220 is generally the location where the reaction medium 300 (e.g., aqueous reaction medium with reactants, products, and/or suspended microorganisms) is present during operation of the bioreactor 200. The bioreactor 200 can further include a headspace portion 230 of the reaction vessel 210 and a bioreactor gas outlet 234 in fluid communication with the headspace portion 230. The headspace portion 230 is generally the upper portion of the reaction vessel 230, which portion is not occupied by liquid reaction medium 300 during bioreactor 200 operation and contains metabolic product gas(es) produced by the biofilms 136, 146 and/or in the liquid reaction medium 300 (e.g., via suspended microorganisms). The metabolic product gas(es) so produced have sufficient residence time within the bioreactor 200 to enter the gas phase in the headspace 230 (e.g., attaining equilibrium or near equilibrium). In particular, relatively higher production of methane or other gas products at the biofilm surface combined with recirculation within the bioreactor 200 provides time for gas product equilibration in the liquid phase (i.e., where methane or other gas products can be originally produced at above-equilibrium concentrations in the biofilm and neighboring reaction medium) and release/capture of gaseous products in the headspace 230. This increases methane or other gas product yield/recovery and reduces (undesirable) gas product loss in the bioreactor 200 effluent, which can represent both a loss of desired product and a potential environmental pollutant (e.g., in the case of methane as a greenhouse gas). The gas outlet 234 is for removal and recovery of any metabolic gas products produced in the bioreactor 200, for example including methane or other gas products.

In an embodiment, the bioreactor insert apparatus 100 can be rotatably mounted within the reaction vessel 210. During operation of the bioreactor 200, rotation of the bioreactor insert apparatus 100 can help to create a well-mixed bulk reactor medium 300 external to the rotating apparatus 100 and within the reaction vessel 200. In this case, an axisymmetric geometric design for the apparatus 100 as generally illustrated in FIGS. 1-5 provides an additional benefit in that the radial mesh/biofilm arms 130, 140 provide not only an increased surface area for biological reaction, but also an impeller-type structure for bulk reactor medium 300 shearing at a rate that does not damage the microorganisms, but which still provides mixing. Rotation of the apparatus 100 can be performed in a bioreactor 200 with a single or multiple apparatus 100 rotatably mounted therein, and/or with a single or multiple first/second structures 102, 104 rotatably mounted therein. For example, as illustrated in FIG. 5, overall bioreactor 200 feed enters into the inlet volume 110 of the first structure 102 and flows radially outward into the exit volumes 130, through meshes/biofilms 134, 136 thereon, and into the bulk reaction medium 300. Mixed fluid from the bulk reaction medium 300 then enters the recirculation volumes 140 on the second structure 104 in the bioreactor vessel 210, through meshes/biofilms 144, 146, into the outlet volume 120 of the second structure 104, and then out through the first fluid outlet 124 (e.g., for partial recycle and/or product withdrawal). The two structures 102, 104 (or multiple unitary apparatus 110 as in FIGS. 1-4) can be independently rotatable to provide mixing of the bulk reaction medium 300.

In an embodiment, the bioreactor 200 can further include a membrane filtration unit 401 in fluid communication with an outlet or effluent stream of the bioreactor 200, for example a non-recycled portion of the effluent from the first fluid outlet 124. In various embodiments, the membrane filtration unit 401 can include one or more of the following: a separation membrane, a membrane inlet 402 in fluid communication with the first fluid outlet 124 and a first (retentate) side of the separation membrane 401, a membrane retentate outlet 404 in fluid communication with the first fluid inlet 114 and the first (retentate) side of the separation membrane 401, and a membrane permeate outlet 406 in fluid communication with a second (permeate; opposing) side of the separation membrane 401 (e.g., as final, clarified effluent stream). The separation membrane can be a semi-permeable membrane adapted to retain solids and to selectively transmit or retain gases, liquids, and solutes therein based on size, solubility, ionic or non-ionic character, etc. as determined by membrane pore size, structure, chemical constituents, etc., as generally known in the art. The membrane generally retains bioreactor (microorganism) solids on the retentate side and transmits methane or other (dissolved) gaseous products, or other dissolved non-gaseous compounds, and water on the permeate side. The retentate can be recycled back to the bioreactor 200/bioreactor insert apparatus 100.

In another aspect, the disclosure relates to a method for forming a bioreactor product. The method comprises: providing a bioreactor and bioreactor insert apparatus according to any of their variously disclosed embodiments, wherein: the bioreactor insert apparatus further comprises biofilms adhered to the biofilm supports, the biofilms having biological (e.g., methanogenic) activity, an aqueous reaction medium at least partially fills the interior reaction volume and the bioreactor insert apparatus, and suspended microorganisms are present in the aqueous reaction medium; feeding a thermal fluid (or working fluid) having a selected (e.g., controlled) temperature through the conduits mounted adjacent to the biofilm supports (e.g., exit conduits adjacent to the exit biofilm supports, recirculation conduits adjacent to the recirculation biofilm supports, etc.), thereby maintaining (or controlling) the temperature of the biofilm supports and biofilms thereon at a pre-selected temperature or within a temperature range (e.g., a substantially uniform temperature or an induced temperature gradient); feeding an influent stream (e.g., wastewater influent) comprising one or more reactants for conversion to the bioreactor insert apparatus via the first fluid inlet; circulating the influent stream through the inlet volume, into the exit volumes, forced through the exit biofilm supports and biofilms thereon, into the external volume, forced through the recirculation biofilm supports and biofilms thereon, into the recirculation volumes, into the outlet volume, and through the first fluid outlet; and converting the one or more influent reactants to a (e.g., methane) product, in particular by biofilm activity and/or suspended microorganism activity.

An influent stream (e.g., wastewater influent) including one or more organic constituents or other reactants for biological conversion is fed to the bioreactor insert apparatus 100 via the first fluid inlet 114, such as with suitable pumping apparatus and/or suitable valves, piping, or other fitting structures. The bioreactor 200 first fluid inlet 114 further can receive and deliver into the bioreactor insert apparatus 100 a recycle stream of effluent from the first fluid outlet 124. Influent feed and recycle can be mixed together upstream of the first fluid inlet 114 and delivered into the apparatus 100 together, or they can be fed separately to the apparatus 100 at different fluid inlet locations. The influent stream is circulated through the inlet volume 110, into the exit volumes 130, through the exit biofilm supports 134 and biofilms 136 thereon, into the external volume 150 (e.g., into the reaction medium 300, through the recirculation biofilm supports 144 and biofilms 146 thereon), into the recirculation volumes 140, into the outlet volume 120, and through the first fluid outlet 124 (e.g., including suitable valves, piping, and/or other fitting structures). In FIG. 1, the arrows without reference numerals qualitatively indicate the direction of flow and circulation through the insert apparatus 100 and the bioreactor 200. During the circulation, one or more of the organic constituents or other reactants are converted to a product, both at the biofilm surfaces 136, 146 and in the aqueous reaction medium 300 bulk from suspended microorganisms therein. In an embodiment, the wastewater influent stream includes one or more organic constituents and other reactants for conversion (e.g., anaerobic, anoxic, or aerobic conversion), such as to methane or other product. Wastewater treatment refers to the process by which polluted water sources from domestic sources (sanitation) or industrial sources are converted into a clean effluent that can be returned to natural sources or used for other applications (e.g., irrigation). The disclosed bioreactor 200 and insert apparatus 100 can be used in wastewater treatment for the treatment of such contaminated water, for example while also producing a methane product. Wastewater influent streams can include a variety of organic constituents such as proteins, lipids, and/or carbohydrates. Examples of other wastewater components that serve as microorganism nutrients for metabolic conversion in the bioreactor 200 include ammonium ions (e.g., for aerobic conversion to nitrite and nitrate ions or salts), nitrate ions (e.g., for anoxic conversion to nitrogen gas), and phosphate ions (e.g., for conversion to polyphosphate for storage in microbes which are then collected).

In an embodiment, at least 50%, 65%, or 80% of total microorganisms in the bioreactor 200 are incorporated into the biofilms, for example as determined on a weight or number basis. Suitably, the remaining microorganisms are suspended in the bulk reaction medium 300 and/or within the insert apparatus 100, for example with the bulk reaction medium 300 and insert apparatus 100 having generally the same or similar concentrations of suspended microorganisms. Suitably at least 50, 65, 80, 85, 90, 95, or 98% and/or up to 90, 95, 97, 98, 99, or 100% of total microorganisms are incorporated into the biofilms 136, 146 with only a minor amount in the bulk reaction medium 300 and/or insert apparatus 100. Total biological activity in the bioreactor likewise can be at least 80, 85, 90, 95, or 98% and/or up to 90, 95, 97, 98, 99, or 100% attributed to biological activity/conversion at the biofilm 136, 146 surfaces. In some embodiments, the exit supports 134 buildup highly active biofilms 136 and are responsible for a majority of product formation (e.g., methane production in anaerobic configurations). In such cases, the biofilms 146 of the recirculation supports 144 are predominantly useful for solid/liquid separation, limiting solids in the reaction medium 300 from reentering the insert apparatus 100 via the recirculation volumes 140, although potentially still providing some biological activity. In some embodiments, the recirculation supports 144 buildup highly active biofilms 146 and are responsible for a majority of product formation (e.g., methane production in anaerobic configurations). In such cases, the biofilms 136 of the exit supports 134 are predominantly useful for solid/liquid separation, limiting solids in the reaction medium 300 from reentering the insert apparatus 100 via the exit volumes 130, although potentially still providing some biological activity. In some embodiments, the exit supports 134 and the recirculation support 144 buildup highly active biofilms 136, 146, which both contribute to product formation and/or solid/liquid separation.

In an embodiment, the biofilms 136, 146 have methanogenic activity. At least some microorganisms in the biofilm 136, 146 (and bioreactor 200) are methanogens, producing methane from one or more components in the influent feed and/or as produced by other microorganisms as intermediates such as organic acid intermediates. Suitably, aqueous fluid removed through the first fluid outlet 124 includes dissolved methane at a concentration in a range of 50% to 150% relative to the equilibrium concentration of methane in water, for example as determined by Henry's law (e.g., at the temperature and/or pressure conditions of the first fluid outlet 124). For example, the relative methane concentration can be at least 50, 70, 80, 90, 95, 99, or 100% and/or up to 110, 120, 135, or 150% relative to the equilibrium concentration of methane in water. The method can further include recovering a gas product stream including methane via the bioreactor gas outlet 234, for example containing about 60-95% methane, with the balance being substantially carbon dioxide and hydrogen.

In an embodiment, the method comprises operating the bioreactor at a temperature in a range of 5° C. to 30° C., 15° C. to 30° C. or 20° C. to 25° C. In some cases, the bioreactor can be operated at a mesophilic temperature such as 20° C. to 40° C., for example at least 20, 25, or 30° C., and/or up to 30, 35, or 40° C. In some cases, the bioreactor can be operated at a thermophilic temperature such as 40° C. to 60° C., for example at least 40, 45, or 50° C., and/or up to 50, 55, or 60° C. The foregoing ranges are typical of wastewater treatment, for example representing the temperature of the reaction medium 300 and the fluid in the insert apparatus 100. More generally, any suitable temperatures can be used for other applications, whether for wastewater treatment or otherwise, in particular as long as such temperatures do not damage, kill, or otherwise inactivate the useful microorganisms in the biofilms. For example, reactor temperature can be used as a selection pressure to inactivate certain microbes, for example methanogen inactivation in a scenario where it is desired to select for spore-forming microbes that produce hydrogen as preferred product. These temperatures can represent the ambient or bulk fluid temperature of the bioreactor, such as the temperature of the influent stream and/or the temperature of the liquid reaction medium of the in the interior reaction volume or the reaction vessel, for example including a range of time-dependent operating temperatures resulting from (naturally) varying influent temperatures (e.g., in which cases the thermal management conduits can still maintain desired biofilm/biofilm support operating temperatures).

In an embodiment, the bioreactor can be operated over a relatively wide range of hydraulic retention time (HRT) values, in particular at relatively lower values that reflect its ability to operate at relatively high volumetric loading rates while still providing good microorganism activity. The HRT suitably can range from 2 hr to 40 hr, for example being at least 2, 5, 8, 10, 12, 15, or 20 hr and/or up to 10, 15, 20, 25, 30, or 40 hr. The HRT can be expressed as the ratio V_{R}/Q reflecting the bioreactor 200 volume (V_{R}) relative to the inlet 114/114A volumetric flow rate (Q) to the bioreactor 200.

### EXAMPLES

The following examples illustrate a bioreactor insert apparatus, corresponding bioreactor, and operation of the same according to the disclosure.

In order to understand the configuration required to support the radiator design, mathematical analysis was conducted to model how the heat travels along the mesh from a pipe. This was done using an understanding of thermodynamics, heat transfer, and differential equations.

A series of assumptions were made to complete the mathematical model. To understand the temperature gradient of the mesh, it was assumed that the temperature at the contact point between the pipe and mesh is a known constant point heat source. This assumption was reasonable for this analysis, which was focused only on the temperature structure of the mesh and excluded the pipe. In addition, it was assumed that the surrounding water is at a fixed temperature, behaving as a constant heat sink due to the bulk recirculation system. The bulk recirculation system also caused the convection to be small enough that the convection between the mesh and surrounding water could be ignored. The radiation in this system was also assumed to be insignificant. As the mesh conducts the majority of the heat, the thermal conductivity of the biofilm was ignored. And, due to the very small thickness of the biofilm, perfect thermal contact between the mesh and biofilm was assumed, therefore assuming that the biofilm is the same temperature as the mesh. Lastly, it was assumed that the temperature stops changing after a defined length, which can be determined empirically.

To understand how this system behaves, the following equations were utilized: the Contact Resistance equation (1), Fourier's equation (2) (3) and its derivative (4), and the Temperature Difference equation (5) and its second derivative (6).

$${q}_{c}^{′} = {h}_{c} ( T - {T}_{a} ) dx \qquad ( 1 )$$

$${q}_{x}^{′} = - kt \frac{dT}{dx} \qquad ( 2 )$$

$${q}_{x + dx}^{′} = {q}_{x}^{′} + \frac{d {q}_{x}^{′}}{dx} dx \qquad ( 3 )$$

$$\frac{d {q}_{x}^{′}}{dx} = \frac{d}{dx} [ - kt \frac{dT}{dx} ] \qquad ( 4 )$$

$$θ = T - {T}_{a} \qquad ( 5 )$$

$$\frac{{d}^{2} θ}{{dx}^{2}} = \frac{{d}^{2} T}{{dx}^{2}} \qquad ( 6 )$$

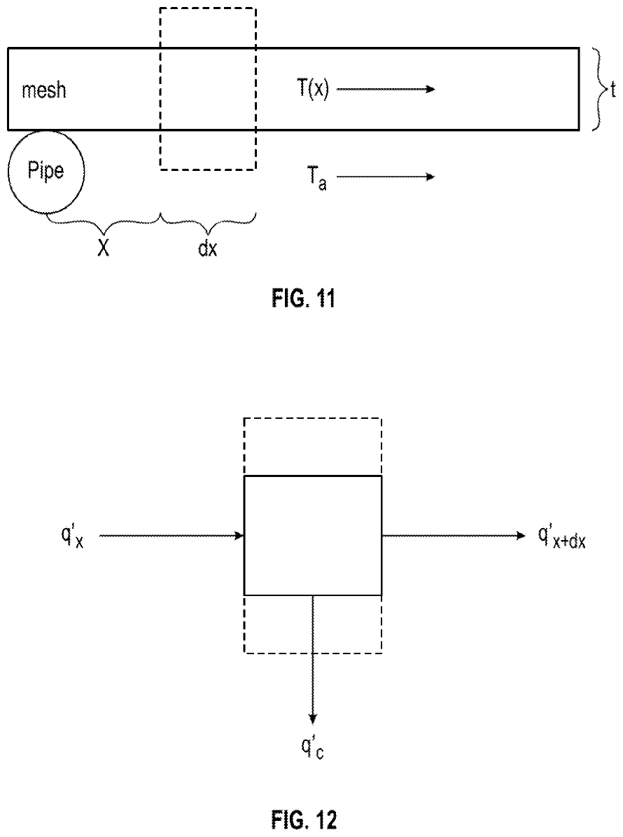

The analysis was modeled in FIG. 11 where the pipe and mesh are shown with the temperature gradient represented by T(x) and the ambient water temperature represented by T_{g}. The thickness of the pipe is t and a control volume of length dx, at distance x away from the pipe was chosen for analysis. A conservation of energy equation (7) was derived from the control volume setup in FIG. 12. This equation, in combination with equations 1-6 led to the differential equation (8).

$${q}_{x}^{′} = {q}_{x + d x}^{′} + {q}_{c}^{′} \qquad ( 7 )$$

$$\frac{{d}^{2} θ}{d {x}^{2}} = \frac{{h}_{c}}{k t} θ \qquad ( 8 )$$

To solve this equation, two initial conditions were defined, the first being that when distance away from the pipe is zero the mesh is the same temperature as the pipe, and the second being that the temperature stops changing some distance L away from the pipe. A temperature gradient equation (9) was derived.

$$θ ( x ) = \frac{{θ}_{0} {e}^{\sqrt{\frac{{k}_{c}}{k t}} x}}{{e}^{\sqrt[2]{\frac{{k}_{c}}{k t}} L} + 1} + \frac{{θ}_{0} {e}^{- \sqrt{\frac{{k}_{c}}{k t}} x}}{1 - {( {e}^{\sqrt[2]{\frac{{k}_{c}}{k t}} L} + 1 )}^{- 1}} \qquad ( 9 )$$

To model the two pipe configuration, it was expected that two super positioned gradients from the one pipe model will be able to predict the gradient of the two pipe system. The mathematical model based on this assumption is shown in Equation 10, where the two one pipe gradients were combined. Here, w is the distance between the two pipes.

$$θ ( x ) = \frac{{θ}_{0} {e}^{\sqrt{\frac{{k}_{c}}{k t}} x}}{{e}^{\sqrt[2]{\frac{{k}_{c}}{k t}} L} + 1} + \frac{{θ}_{0} {e}^{- \sqrt{\frac{{k}_{c}}{k t}} x}}{1 - {( {e}^{\sqrt[2]{\frac{{k}_{c}}{k t}} L} + 1 )}^{- 1}} + \frac{{θ}_{0} {e}^{\sqrt{\frac{{k}_{c}}{k t}} ( ω - x )}}{{e}^{\sqrt[2]{\frac{{k}_{c}}{k t}} L} + 1} + \frac{{θ}_{0} {e}^{- \sqrt{\frac{{k}_{c}}{k t}} ( ω - x )}}{1 - {( {e}^{\sqrt[2]{\frac{{k}_{c}}{k t}} L} + 1 )}^{- 1}} \qquad ( 10 )$$

The empirical analysis focused on validating the selected concept and providing quantifiable results on its performance. The goal of the empirical analysis was to confirm the validity of the mathematical model developed. Three iterations of empirical analysis were performed, each with some number of copper pipes running hot water underneath a large piece of stainless steel biofilm mesh.

The experimental setup delivered hot water from a sink faucet, through the copper pipes, under a simulated biofilm and recorded the temperatures of the influent thermal fluid, effluent thermal fluid, bulk fluid temperature, and simulated biofilm. The goal of the experimental design was to model full-scale wastewater treatment as closely as possible. To accomplish this goal, four main subsystems were included: the thermal sensor array, mock reactor, working fluid line, and bulk recirculation system.

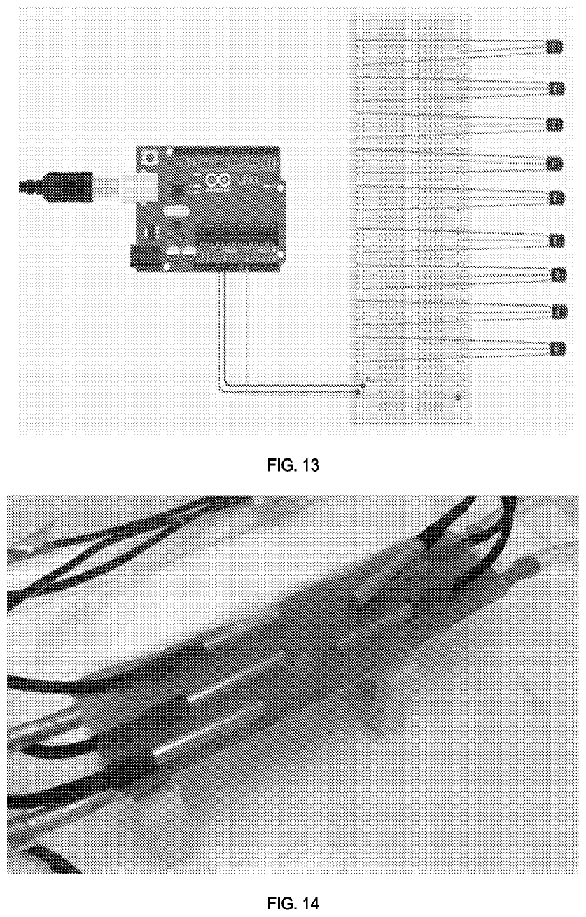

Nine thermal sensors were placed throughout the entire experimental setup. The thermal sensors were fed into a breadboard to form a single wire bus that interfaced with an Arduino Mega as shown in FIG. 13.

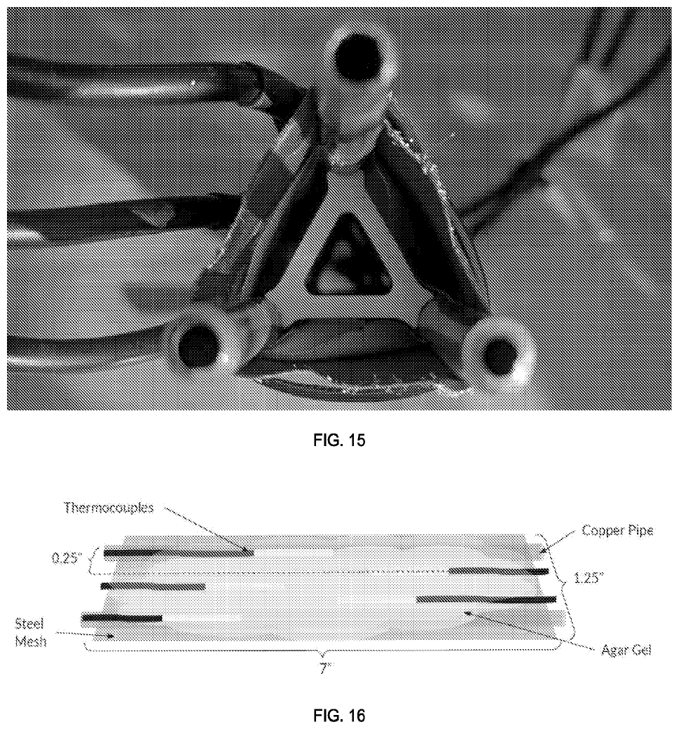

The mock reactor consisted of the simulated biofilm assembly and a plastic tub. The one and two pipe iterations of the proof of concept consisted of two copper pipes spanned by a thin section of mesh as shown in FIG. 14. For the one pipe system, thermal fluid was only passed through one of the two pipes in the system. For the three pipe system, the mesh was wrapped around three pipes spaced using an internal 3D printed structure, shown in FIG. 15. Both systems were placed on a set of 3D printed brackets to space them off the floor of the tub during testing. As biofilms can take up to 200 days to grow, the biofilm was simulated through agar gel because of its similar relevant thermal properties to biofilm. This agar gel was poured and allowed to solidify on top of the mesh (or on one side of the triangle in the case of the three pipe system) with five sensors resting on the mesh positioned at quarter-inch intervals away from the pipe, with the first one placed directly over the pipe. This layout is shown in FIG. 16. Additionally, one sensor was laid directly on top of the biofilm once it was set in order to assess the heat transfer through the agar gel, as shown in FIG. 14.

Water was selected as the thermal fluid as it is easy to obtain, has a high specific heat, has a low viscosity, and doesn't present any major shortcomings or challenges. The thermal fluid line started at a sink faucet and used several fittings to adapt to vinyl tubing. Vinyl tubing was selected to transport the water from the faucet to the mock reactor as it can be easily fit to barbs, be easily routed to different locations, and has good thermal insulation. From the faucet, the vinyl tubing flowed into a t-junction. One of the two outputs is routed into a measuring cup to create a reservoir of influent thermal fluid, so its temperature could be measured. The other output was routed back into vinyl tubing that traveled to the simulated reactor. As that tubing passed through the reactor, it was transferred to sections of copper pipe that were attached directly underneath the mesh. Copper was chosen for the pipe material as it was expected to perform well due to its exceptional thermal properties. Furthermore, it is readily available in a selection of convenient pipe diameters and lengths. After passing through the copper pipes, the working fluid was then routed back into the tubing and back to the sink. This effluent line is also routed into a measuring cup with the matching effluent thermal fluid sensor submerged, as was done with the influent.

The bulk recirculation system aimed to keep the water in the reactor at a constant level and continuously moving in order to keep the entirety of the bulk fluid in the reactor at a uniform temperature. Clean water was used as the bulk fluid because it has similar thermal properties to wastewater, which was the only important similarity required for this proof of concept. This subsystem consisted of a separate bucket, pump, and two rubber tubes. The pump was placed in the bucket and submerged in bulk fluid. One of the tubes was used to pump the bulk fluid from the bucket into the mock reactor, and the other acted as a drainage from the reactor back into the bucket underneath the reactor. One of the thermal sensors was located in the reactor's bulk fluid to record the uniform ambient temperature.

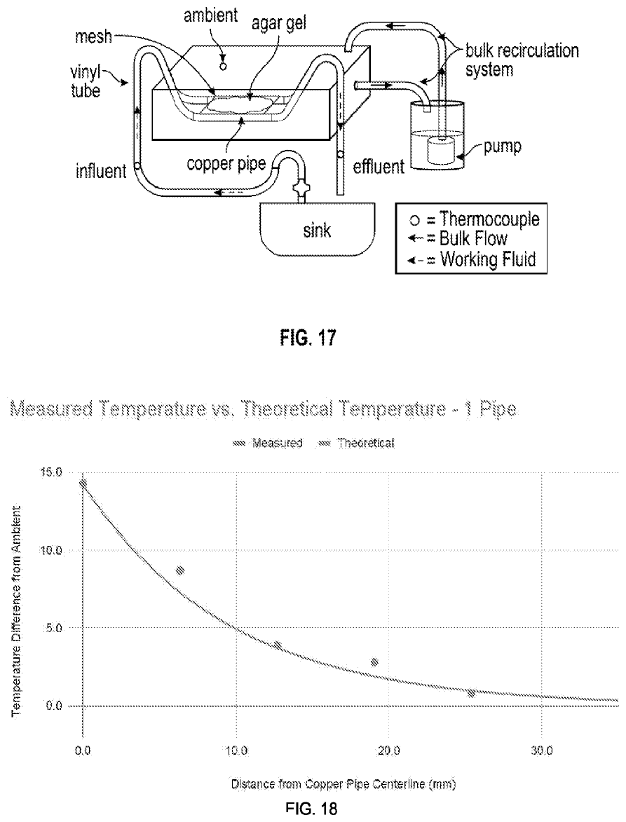

The setup and interaction of the four major subsystems can be seen below in FIG. 17. In order to create this setup, some aspects of the design required manufacturing. A drill was used to create a drainage hole for the bulk recirculation system. The vinyl and copper tubing were cut with a pipe cutter. The mesh was cut to size with a coping saw. Epoxy was used to attach the mesh to the copper pipes. The agar gel was made as specified in the instructions that were provided by the manufacturer, which consisted of boiling water and mixing a ratio of water to agar. Once the setup was built and connected to the selected faucet, the experimental procedure was followed. The procedure began by turning on the hot side of the faucet at a low level. This created the maximum thermal fluid temperature without overloading the system with too much pressure. Next, the bulk fluid recirculation pump was turned on, and the system was allowed to stabilize for three minutes. This step is essential because without allowing the system adequate time to reach steady state, the amount of fluid in the mock reactor and the temperature of the mesh would still be changing. After the system was given enough time to reach steady state, the data was recorded. During this one-minute period, the Arduino logged the temperature of all nine thermocouples every second. After the one-minute trial was over and the Arduino program was shut down, the faucet and bulk fluid recirculation pump were shut off. The excess bulk water was then drained into the bulk fluid bucket and the mesh, biofilm, and copper pipe were allowed to cool to ambient temperature.

The data from the temperature logs for each pipe configuration was analyzed for comparison with the completed mathematical model.

One Pipe. Table 1 displays the temperature results of the influent, effluent, and ambient thermocouple readings averaged over one minute. Table 2 displays this information for the five thermocouple locations in the biofilm as a function of their distance from the copper pipe. The temperature difference from the ambient temperature was also included in this table, as it is used for plotting.

TABLE 1

| | Average Temperature (° C.) | Standard Deviation |
|---|---|---|
| Influent | 53.7 | 0.0 |
| Effluent | 52.2 | 0.1 |
| Ambient | 24.6 | 0.0 |

TABLE 2

| Distance | Average | | Temperature |
|---|---|---|---|
| from Copper Pipe | Temperature | Standard | Difference from |
| Centerline (mm) | (° C.) | Deviation | Ambient |
| 00.00 | 28.9 | 0.2 | 14.3 |
| 06.35 | 33.8 | 0.8 | 08.7 |
| 12.70 | 28.5 | 0.5 | 03.9 |
| 19.05 | 27.4 | 0.4 | 02.8 |
| 25.40 | 25.40 | 0.1 | 00.8 |

Based on the empirical data, the difference in temperature of the ambient water and the measured temperature where the pipe contacted the mesh, Oo, was calculated to be 14.3° C. Additionally, the thermal contact resistance between stainless steel and water, h_{c}, was found to be 0.000335 W/mmK and the thermal conductivity of steel, Kₛₜₑₑₗ, was found to be 0.015 W/mmK. From the data, a distance of 1 inch away from the copper pipe centerline was chosen to be the length at which the temperature stops changing. Finally, the thickness of the mesh and biofilm was estimated to be 2 mm.

From these characteristics, the theoretical model was plot against the measured data to produce the graph in FIG. 18, which shows a strong correlation between the experimental data and the mathematical model. This figure demonstrates the relationship between the measured temperature from the experiment and the theoretical temperatures as determined by the mathematical analysis.

Two Pipe. Table 3 displays the temperature results of the influent, effluent, and ambient thermocouple readings averaged over one minute. Table 4 displays this information for the five thermocouple locations in the biofilm as a function of their distance from the copper pipe. The temperature difference from the ambient temperature was also included in this table as it is used for plotting.

TABLE 3

| | Average Temperature (° C.) | Standard Deviation |
|---|---|---|
| Influent | 53.0 | 0.1 |
| Effluent | 50.5 | 0.3 |
| Ambient | 25.8 | 0.1 |

TABLE 4

| Distance | Average | | Temperature |
|---|---|---|---|
| from Copper Pipe | Temperature | Standard | Difference from |
| Centerline (mm) | (° C.) | Deviation | Ambient |
| 00.00 | 38.1 | 1.0 | 12.3 |
| 06.35 | 34.3 | 0.4 | 08.5 |
| 12.70 | 31.9 | 0.4 | 06.1 |
| 19.05 | 34.8 | 0.5 | 09.0 |
| 25.40 | 37.7 | 0.8 | 011.9 |

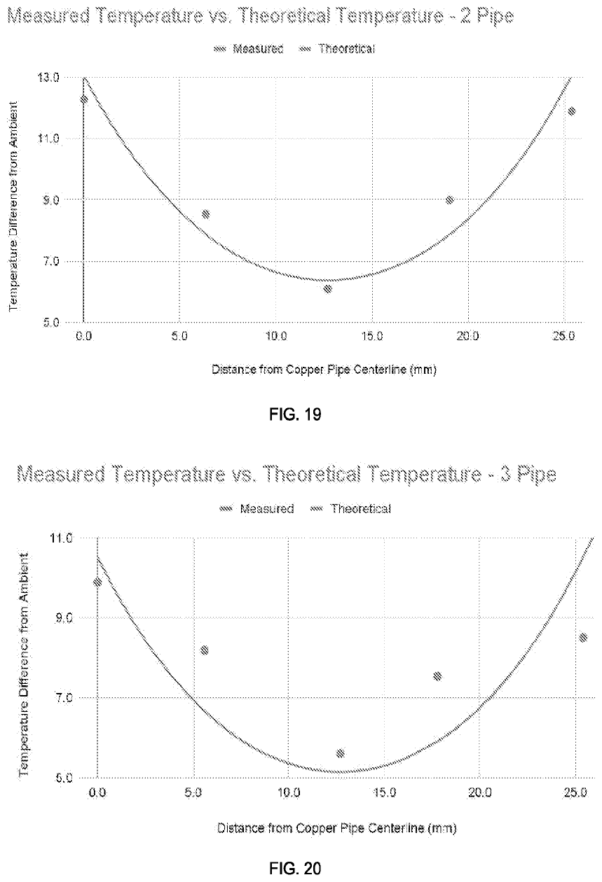

The same characteristics used for the one pipe configuration were also used to create the plot in FIG. 19, except that Oo was found to be 12.3° C. in the two pipe configuration. There is a strong correlation between the data and the mathematical model. FIG. 19 demonstrates the relationship between the measured temperature from our experiment and the theoretical temperatures as determined by our mathematical analysis.

Three Pipe. Table 5 displays the temperature results of the influent, effluent, and ambient thermocouple readings averaged over one minute. Table 6 displays this information for the five thermocouple locations in the biofilm as a function of their distance from the copper pipe. The temperature difference from the ambient temperature was also included in this table, as it is used for plotting.

TABLE 5

| | Average Temperature (° C.) | Standard Deviation |
|---|---|---|
| Influent | 53.4 | 0.1 |
| Effluent | 51.9 | 0.1 |
| Ambient | 25.5 | 0.1 |

TABLE 6

| Distance | Average | | Temperature |
|---|---|---|---|
| from Copper Pipe | Temperature | Standard | Difference from |
| Centerline (mm) | (° C.) | Deviation | Ambient |
| 00.00 | 35.4 | 0.1 | 09.9 |
| 06.35 | 33.7 | 0.1 | 08.2 |
| 12.70 | 31.1 | 0.0 | 05.6 |
| 17.70 | 33.0 | 0.2 | 07.5 |
| 25.40 | 34.0 | 0.0 | 08.5 |

The same characteristics used for the one pipe configuration were also used to create the plot in FIG. 20, except that Oo was found to be 9.9° C. in the three pipe configuration. FIG. 20 demonstrates the relationship between the measured temperature from our experiment and the theoretical temperatures as determined by the mathematical analysis.

As can be seen by the plots, the data supports the mathematical models developed. The one and two pipe data fit the theoretical equation very well, while the three pipe plot has a little more variation. It is believed that this variation is caused by the effects of superpositioning and the triangle shape used for the mesh. A sixth thermocouple was left inside the triangular mesh for the three pipe configuration, which recorded a 5.3° C. temperature difference above ambient. This may be explained by heated bulk fluid being trapped inside the triangular mesh and therefore transferring more heat to the mesh. This would cause the mesh between the pipes to experience a greater rise in temperature due to convection. These tests validated the mathematical model.

The relationship between the number of pipes and the amount of heat transfer through the system was empirically calculated by measuring the volumetric flow rate from the pipe during the experiment and dividing by the number of pipes that the water was being split between in order to calculate the per pipe volumetric flow rate, which was then converted to a mass flow rate. Then, in combination with the measured temperature difference and the specific heat capacity of water the heat transfer was calculated for each pipe setup and the results are shown in Table 7 below. Here, the amount of heat transfer per pipe decreased as pipes were added, which was expected as it was predicted that the heat from the pipes would combine to keep the system warm with less heat transfer per pipe.

TABLE 7

| | | Pipe | Mass | | Heat |
|---|---|---|---|---|---|
| | Volumetric | Volumetric | Flow | Temperature | Transfer |
| Number of | Flow Rate | Flow Rate | Rate | Difference | per |
| Pipes | (cups/min) | (cups/min) | (g/s) | (° C.) | Pipe (W) |
| 1 Pipe | 4.9 | 4.9 | 19.1 | 1.5 | 123 |
| 2 Pipe | 5.3 | 2.6 | 10.4 | 2.5 | 109 |
| 3 Pipe | 7.5 | 2.5 | 9.9 | 1.6 | 65 |

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expressions “coupled,” “connected,” and “communication,” along with their derivatives. For example, some embodiments may be described using the terms “coupled” or “in communication” to indicate that two or more elements are in direct physical or electrical contact. The terms “coupled” or “in communication,” however, may also mean that two or more elements are not in direct contact with each other, but yet still cooperate or interact with each other, for example with fluids (e.g., liquid and/or gas, optionally with dispersed solids therein) being able to flow between the elements, such as via suitable piping or other conduits. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as an example only and does not describe every possible embodiment, as describing every possible embodiment would be impractical, if not impossible. One could implement numerous alternate embodiments, using either current technology or technology developed after the filing date of this application.

### Figure Parts List

100 bioreactor insert apparatus
102 first (or inlet/influent/feed) structure
104 second (or outlet/effluent/permeate) structure
110 inlet volume
112, 112A cylindrical tube, tube axis/centerline
114, 114A first fluid inlet, fresh feed inlet
120 outlet volume
122, 122A annular or cylindrical tube, tube axis/centerline
124, 124A first fluid outlet, recycle portion of effluent
130 exit volumes
132 exit tubes
134 exit biofilm support (e.g., mesh)
136 exit biofilm
138 exit biofilm seed/precursor
140 recirculation volume
142 recirculation tubes
144 recirculation biofilm support (e.g., mesh)
146 recirculation biofilm
148 recirculation biofilm seed/precursor
150 external volume
200 bioreactor
210, 210A, 210B reaction vessel
220 interior reaction volume
230 headspace
234 gas outlet
300 reaction medium
400 exit conduit
401 separation membrane
402, 404, 406 membrane inlet, retentate outlet, permeate outlet
410 recirculation conduit
420 thermal fluid inlet
430 thermal fluid outlet
440 conduit interior volume

1. A bioreactor insert apparatus for reaction medium circulation, biofilm support, and biological treatment, the apparatus comprising:
an inlet volume in fluid communication with a first fluid inlet;
at least one exit volume, each exit volume being in fluid communication with the inlet volume and comprising at least one exit biofilm support disposed at a boundary between the exit volume and an external volume outside the bioreactor insert apparatus, wherein: the exit biofilm support is adapted to promote growth, attachment, and metabolism of microorganisms in the form of a biofilm thereon, and the exit biofilm support is adapted to permit fluid and solid transport across the exit biofilm support and between the exit volume and the external volume; and
at least one exit conduit mounted adjacent to the exit biofilm support, wherein:
each exit conduit comprises a thermal fluid inlet, a thermal fluid outlet, and an interior volume adapted to receive and permit flow of a thermal fluid from the thermal fluid inlet to the thermal fluid outlet via the interior volume;
the exit conduit interior volume is substantially liquidly isolated from the first fluid inlet, the inlet volume, the exit volume, and the external volume; and
the exit conduit is adapted to provide heat transfer between a thermal fluid in the exit conduit and the exit biofilm support, thereby controlling a temperature of the exit biofilm support.

2. The apparatus of claim 1, wherein the exit conduit comprises a metal material.

3. The apparatus of claim 1, wherein the exit conduit comprises a thermally insulating material for one or more regions of the exit conduit that are not adjacent to the biofilm support.

4. The apparatus of claim 1, wherein the exit conduit comprises a semi-permeable material adapted to permit selective transport of a substance in the thermal fluid through the semi-permeable material.

5. The apparatus of claim 1, wherein the exit conduit is in fluid communication with one or more of a heater, cooler, and pump for circulation of a temperature-controlled thermal fluid through the exit conduit.

6. The apparatus of claim 5, wherein:
the exit conduit is in fluid communication with the heater and the cooler; and
the exit conduit is adapted to cycle between heating and cooling states to expand and contract the biofilm support and any biofilm thereon.

7. The apparatus of claim 1, wherein the exit conduit is in fluid communication with a reservoir containing the thermal fluid therein.

8. The apparatus of claim 7, wherein the thermal fluid comprises water.

9.-15. (canceled)

16. The apparatus of claim 1, wherein the exit conduit is adapted to maintain the biofilm support at a temperature within ±6° C. of a selected setpoint temperature.

17. The apparatus of claim 1, wherein the exit conduit is adapted to maintain a temperature gradient in the biofilm support with at least two different setpoint temperatures that are at least 2° C. different from each other.

18. The apparatus of claim 1, wherein each biofilm support is adjacent to a plurality of exit conduit segments at spaced-apart locations of the biofilm support.

19. The apparatus of claim 1, wherein the exit conduit is positioned at least partially within the exit volume.

20. The apparatus of claim 1, wherein the exit conduit is positioned at least partially within the external volume.

21. The apparatus of claim 1, wherein the exit conduit is positioned at least partially within the exit volume and the external volume.

22. The apparatus of claim 1, further comprising:
an outlet volume in fluid communication with a first fluid outlet;
at least one recirculation volume, each recirculation volume being in fluid communication with the outlet volume and comprising at least one recirculation biofilm support disposed at a boundary between the recirculation volume and the external volume outside the bioreactor insert apparatus, wherein: the recirculation biofilm support is adapted to promote growth, attachment, and metabolism of microorganisms in the form of a biofilm thereon, and the recirculation biofilm support is adapted to permit fluid and solid transport across the recirculation biofilm support and between the exit volume and the external volume; and
optionally at least one recirculation conduit mounted adjacent to the recirculation biofilm support, wherein:
each recirculation conduit comprises a thermal fluid inlet, a thermal fluid outlet, and an interior volume adapted to receive and permit flow of a thermal fluid from the thermal fluid inlet to the thermal fluid outlet via the interior volume;
the recirculation conduit interior volume is substantially liquidly isolated from the first fluid inlet, the first fluid outlet, the inlet volume, the outlet volume, the exit volume, the recirculation volume, and the external volume; and
the recirculation conduit is adapted to provide heat transfer between a thermal fluid in the recirculation conduit and the recirculation biofilm support, thereby controlling a temperature of the recirculation biofilm support.

23. The apparatus of claim 22, comprising:
a plurality of the exit volumes, wherein the exit biofilm support is the only fluid communication pathway between the exit volume and the external volume, such that the exit volume is configured to force circulating liquid from the inlet volume, through the exit biofilm support, and into the external volume; and
a plurality of the recirculation volumes, wherein the recirculation biofilm support is the only fluid communication pathway between the recirculation volume and the external volume, such that the recirculation volume is configured to receive forced circulating liquid from the external volume, through the recirculation biofilm support, and into the outlet volume.

24. The apparatus of claim 1, wherein:
the bioreactor insert apparatus is a single structure comprising the inlet volume, the outlet volume, the plurality of exit volumes, and the plurality of recirculation volumes;
the inlet volume is defined by a cylindrical tube in fluid communication with the first fluid inlet;
the outlet volume is defined by an annular tube around the cylindrical tube in fluid communication with the first fluid outlet;
the exit volumes are defined by exit tubes in fluid communication with the cylindrical tube inlet volume; and
the recirculation volumes are defined by recirculation tubes in fluid communication with the annular tube outlet volume.

25. The apparatus of claim 1, wherein the biofilm support is in the form of a mesh.

26. The apparatus of claim 1, further comprising a biofilm adhered to the biofilm support, the biofilm comprising a community of microorganisms collectively having biological activity.

27. The apparatus of claim 1, further comprising a biofilm seed adhered to the biofilm support, the biofilm seed comprising a water-soluble adhesive matrix and a community of microorganisms as a biofilm precursor distributed throughout the matrix.

28. A bioreactor comprising:
a reaction vessel defining an interior reaction volume; and
a bioreactor insert apparatus according to claim 1 mounted within the reaction vessel;
wherein the external volume corresponds to a portion of the interior reaction volume outside the bioreactor insert apparatus.

29. A method for forming a bioreactor product, the method comprising:
providing a bioreactor according to claim 28, wherein:
the bioreactor insert apparatus further comprises biofilms adhered to the biofilm supports, the biofilms having biological activity,
an aqueous reaction medium at least partially fills the interior reaction volume and the bioreactor insert apparatus, and
suspended microorganisms are present in the aqueous reaction medium;
feeding a thermal fluid having a selected temperature through the conduits mounted adjacent to the biofilm supports, thereby maintaining the temperature of the biofilm supports and biofilms thereon at a pre-selected temperature or within a temperature range;
feeding an influent stream comprising one or more reactants for conversion to the bioreactor insert apparatus via the first fluid inlet;
circulating the influent stream through the inlet volume, into the exit volumes, forced through the exit biofilm supports and biofilms thereon, into the external volume, forced through the recirculation biofilm supports and biofilms thereon, into the recirculation volumes, into the outlet volume, and through the first fluid outlet; and
converting the one or more influent reactants to a product.

30.-36. (canceled)
